(19) United States
(12) Patent Application Publication
(10) Pub. No.: US 2013/0251710 A1
(43) Pub. Date: Sep. 26, 2013

(54) Methods to Expand the Eligible Patient Population for HER2-Directed Targeted Therapies

(71) Applicant: NSABP Foundation, Inc., Pittsburgh, PA (US)

(72) Inventors: Soonmyung Paik, Pittsburgh, PA (US); Katherine Pogue-Geile, Pittsburgh, PA (US); Patrick Gavin, Pittsburgh, PA (US)

(73) Assignee: NSABP Foundation, Inc., Pittsburgh, PA (US)

(21) Appl. No.: 13/889,959

(22) Filed: May 8, 2013

Related U.S. Application Data
(63) Continuation-in-part of application No. 13/093,563, filed on Apr. 25, 2011, now abandoned.
(60) Provisional application No. 61/327,460, filed on Apr. 23, 2010.

(51) Int. Cl. C12Q 1/68; A61K 39/395

(52) U.S. Cl. CPC C12Q 1/6886; A61K 39/39558
USPC 424/133.1; 435/6.11; 506/9; 435/7.1

(57) ABSTRACT

The present disclosure provides improved methods for identifying breast cancer patients that receive an increased benefit from the addition of a HER2-targeted therapy, for example adjuvant trastuzumab, to chemotherapy.

### CROSS-REFERENCE TO RELATED APPLICATION

The present application is a continuation-in-part application of U.S. patent application Ser. No. 13/093,563, which was filed on Apr. 25, 2011, which claims the benefit of U.S. Provisional Patent Application Ser. No. 61/327,460, which was filed on Apr. 23, 2010, both of which are incorporated herein by reference in their entirety.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

Currently HER2-targeted therapies such as trastuzumab or lapatinib are only used in the treatment patients diagnosed with HER2 positive breast cancer, which comprise only 15% to 20% of all breast cancer patients. HER2 positivity is defined by either overexpression of HER2 protein, which is determined by immunohistochemical staining (3+ staining score by FDA approved Herceptest assay), or by amplification of the HER2 (ERBB2) gene, which is determined by fluorescence in situ hybridization assay (HER2/CEP17 ratio over 2 using FDA approved PathVysion assay). The current cut-offs for these assays were determined from clinical trials of patients diagnosed with metastatic or advanced breast cancer.

However, in a trial that tested the worth of addition of trastuzumab to adjuvant chemotherapy in the treatment of stage 2 or 3 breast cancer patients (NSABP trial B-31), even patients diagnosed with breast cancer that was classified as HER2 negative using currently used clinical HER2 assays (IHC and FISH) gained significant benefit from trastuzumab (Paik, et al., N Engl. J. Med. 358:1409-1411, 2008). In this study, degree of HER2 gene amplification or protein expression did not have any correlation with the degree of benefit from trastuzumab, directly challenging the use of currently used HER2 clinical assays (IHC and FISH) for selection of patients for adjuvant trastuzumab or other HER2 targeted therapies.

Therefore improved predictive tests for HER2-targeted therapies are clearly required.

### BRIEF SUMMARY OF THE INVENTION

In order to develop better predictive test for HER2 targeted therapies, whole genome (transcriptome) gene expression profiling was performed on tumor specimens collected from patients enrolled in NSABP trial B-31 using microarrays (Agilent and Affymetrix platforms). As a result of this gene expression profiling effort, it was determined that mRNA expression levels of HER2 (ERBB2) itself is a predictor of the degree of benefit from trastuzumab in NSABP trial B-31. In addition, based on findings from NSABP trial B-31, is was determined that a large number of patients diagnosed with breast cancer that are classified as HER2 negative using current generation HER2 assays (IHC and FISH) are expected to derive benefit from trastuzumab or other HER2 targeted therapies. Therefore, the present disclosure details HER2 assays (based on measurement of HER2 mRNA) that provide a significant improvement over currently used HER2 assays (FISH and IHC) as a predictor of the degree of benefit from HER2 targeted therapies in the treatment of breast cancer in an adjuvant setting (stage 2 or 3 breast cancer).

Currently, breast cancer samples are assayed for HER2 protein levels or HER2 gene copy number, and based on this analysis the breast cancer samples are classified as “HER2 positive” or “HER2 negative.” Breast cancers that are classified as “HER2 positive” are candidates for treatment with a HER2-targeted therapy, such as trastuzumab, while those that are classified as “HER2 negative” are not candidates for HER2-targeted therapy. However, the inventors have determined that many breast cancers that are currently classified as “HER2 negative” still receive a therapeutic benefit from HER2-targeted therapies, such as trastuzumab. Therefore, the present disclosure provides improved assays that are more accurate in predicting the benefit from addition of a HER2-targeted therapy to chemotherapy. Breast cancer samples that were classified as “HER2 negative” by the assays previously described and used in the clinic are often classified as “HER2 positive” using the presently described HER2 mRNA assays. Therefore, numerous breast cancer patients that would not have been candidates for treatment with a HER2-targeted therapy based on the assays previously described and used in the clinic can be correctly identified as candidates for treatment with HER2-targeted therapies, such as trastuzumab, thus improving breast cancer patient care.

The present disclosure provides methods of identifying a cancer patient, for example a breast cancer patient, that has an increased benefit from the addition of a HER2-targeted therapy to chemotherapy, comprising assaying a tumor tissue sample from said patient for expression of HER2 mRNA, wherein a normalized HER2 mRNA expression level of about 6.0 or greater is indicative of a cancer patient that has a increased benefit from the addition of a HER2-targeted therapy to chemotherapy. In certain embodiments, normalized HER2 mRNA expression levels of about 6.0 to about 10.5 are indicative of a cancer patient that has an increased benefit from the addition of a HER2-targeted therapy to chemotherapy. In still other embodiments, normalized HER2 mRNA expression levels that are below the levels previously classified as “HER2 positive” are indicative of a cancer patient that has an increased benefit from the addition of a HER2-targeted therapy to chemotherapy. In particular aspects, normalized HER2 mRNA expression levels of about 6.0, about 6.5, about 7.0, about 7.5, about 8.0, about 8.5, about 9.0, about 9.5, about 10.0, or about 10.5 or greater are indicative of a cancer patient that has a increased benefit from the addition of a HER2-targeted therapy to chemotherapy.

In certain aspects of the present disclosure, the HER2-targeted therapy is trastuzumab, while in other aspects of the present disclosure the HER2-targeted therapy is lapatinib. In particular aspects of the present disclosure, the HER2-targeted therapy is combination of trastuzumab and lapatinib. It will be understood to the skilled artisan that other HER2-targeted therapies, either alone or in combination, could be used in conjunction with the teachings of the present disclosure.

The present disclosure also provides a method of identifying a cancer patient that has an increased benefit from the addition of a HER2-targeted therapy to a standard chemotherapy regimen, comprising assaying a tumor tissue sample from said patient for expression of HER2 or a HER2-related mRNA and estrogen receptor or an estrogen receptor-related mRNA, wherein a value outside of a range of a combined normalized HER2 mRNA expression level between about 11.0 and about 15.0 and a normalized estrogen receptor mRNA expression level of about 10.0 and about 12.0 is indicative of a cancer patient that has an increased benefit from the addition of a HER2-targeted therapy to a chemotherapy regimen. In certain embodiments the HER2-related mRNA is a c17orf37 or GRB7 mRNA. In other embodiments the estrogen receptor-related mRNA is a NAT1, GATA3, CA12 or IGF1R mRNA.

Thus, the present disclosure additionally provides methods of treating breast cancer in a patient in need of such treatment, comprising assaying a breast cancer or tumor tissue sample from said patient for expression of HER2 mRNA, and treating the patient with a HER2-targeted therapy and chemotherapy if the results of the assay indicate a normalized HER2 mRNA expression level of about 6.0 or greater.

The present disclosure further provides a method of treating breast cancer in a patient in need of such treatment, comprising assaying a tumor tissue sample from said patient for expression of HER2 or a HER2-related mRNA and estrogen receptor or an estrogen receptor-related mRNA, and treating the patient with a HER2-targeted therapy and a chemotherapy regimen if the results of the assay indicate a value outside of a range of a combined normalized HER2 or HER2-related mRNA expression level between about 11.0 and about 15.0 and a normalized estrogen receptor or estrogen receptor-related mRNA expression level of about 10.0 and about 12.0. In particular embodiments the HER2-related mRNA is a c17orf37 or GRB7 mRNA. In additional embodiments the estrogen receptor-related mRNA is a NAT1, GATA3, CA12 or IGF1R mRNA.

### BRIEF DESCRIPTION OF THE SEVERAL VIEWS OF THE DRAWINGS

FIG. 1. A plot of the log hazard ratio of trastuzumab in B-31 patients in relation to expression levels of HER2 mRNA.

FIG. 2. A plot of the mRNA levels of samples classified as HER2 negative and HER2 positive from the B-31 and B-28 studies.

FIG. 3. A plot showing the correlation between HER2 mRNA expression levels measured by the Nanostring method (nCounter assay) and the QuantigenePlex method.

### DETAILED DESCRIPTION OF THE INVENTION

Based on findings from NSABP trial B-31, a large number of patients diagnosed with breast cancer that are classified as HER2 negative using current generation HER2 assays (IHC and FISH) derived benefit from trastuzumab, a HER2-targeted therapy. Therefore, the present disclosure details HER2 assays (based on measurement of HER2 mRNA) that provide a significant improvement over currently used HER2 assays (FISH and IHC) as a predictor of the degree of benefit from HER2-targeted therapies in the treatment of breast cancer in an adjuvant setting (stage 2 or 3 breast cancer). In order to develop better predictive test for HER2 targeted therapies, whole genome (transcriptome) gene expression profiling was performed on tumor specimens collected from patients enrolled in NSABP trial B-31 using microarrays (Agilent and Affymetrix platforms). As a result of this gene expression profiling effort, it was determined that mRNA expression levels of HER2 (ERBB2) were a more accurate predictor of the degree of benefit from trastuzumab.

Although specific techniques for the quantitation of HER2 mRNA levels are discussed in the Example below, it will be understood by the skilled artisan that any technique currently used for quantitation of mRNA levels can be used in the practice of the present invention.

Therapeutic formulations are provided as pharmaceutical preparations for local administration to patients or subjects. The term “patient” or “subject” as used herein refers to human or animal subjects (animals being particularly useful as models for clinical efficacy of a particular composition). Selection of a suitable pharmaceutical preparation depends upon the method of administration chosen, and may be made according to protocols well-known to medicinal chemists.

The term “pharmaceutically acceptable carrier” includes any and all solvents, dispersion media, coatings, antibacterial and antifungal agents, isotonic and absorption delaying agents, and the like. The use of such media and agents for pharmaceutically active substances is well-known in the art. Except insofar as any conventional media or agent is incompatible with the platinum-based therapeutic agents, its use in the therapeutic compositions is contemplated. Supplementary active ingredients or therapeutic agents can also be used with the platinum-based therapeutic agents.

As used herein, “pharmaceutically-acceptable salts” refer to derivatives of the disclosed compounds wherein one or more components of the disclosed compounds are modified by making acid or base salts thereof. Examples of pharmaceutically-acceptable salts include, but are not limited to: mineral or organic acid salts of basic residues such as amines; alkali or organic salts of acidic residues such as carboxylic acids; and the like. Thus, the term “acid addition salt” refers to the corresponding salt derivative of a component that has been prepared by the addition of an acid. The pharmaceutically-acceptable salts include the conventional salts or the quaternary ammonium salts of the component formed, for example, from inorganic or organic acids. For example, such conventional salts include, but are not limited to: those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds may exist as zwitterions. All forms of the active agents, including free acid, free base, and zwitterions, are contemplated to be within the scope of the present disclosure.

A protein or antibody can be formulated into a composition in a neutral or salt form. Pharmaceutically acceptable salts include the acid addition salts (formed with the free amino groups of the protein), and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

In addition, the disclosed compositions or components thereof can be complexed with polyethylene glycol (PEG), metal ions, or incorporated into polymeric compounds such as polylactic acid, polyglycolic acid, hydrogels, dextran, and the like. Such compositions will influence the physical state, solubility, stability, rate of in vivo release, and rate of in vivo clearance, and are thus chosen according to the intended application.

The dosage unit forms suitable for injectable use include sterile aqueous solutions or dispersions and sterile powders for the extemporaneous preparation of sterile injectable solutions or dispersions. In all cases the form must be sterile and must be suitably fluid. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the disclosed compounds in the required amount in the appropriate solvent with various of the other ingredients enumerated above, as required, followed by filtered sterilization. Generally, dispersions are prepared by incorporating the various sterilized ingredients into a sterile vehicle that contains the basic dispersion medium and the required other ingredients from those enumerated above. In the case of sterile powders for the preparation of sterile injectable solutions, the preferred methods of preparation are vacuum-drying and freeze-drying techniques, which yield a powder of the dosage unit plus any additional desired ingredient from a previously sterile-filtered solution thereof.

In certain aspects the present disclosure encompasses methods of treating or managing cancer, which comprise administering to a patient in need of such treatment or management a therapeutically effective amount of a disclosed composition or dosage unit thereof. In certain embodiments, such a compound or dosage unit is referred to as an active agent. Use of the disclosed compositions in the manufacture of a medicament for treating or preventing a disease or disorder is also contemplated. The present disclosure also encompasses compositions comprising a biologically or therapeutically effective amount of one or more of the disclosed compounds for use in the preparation of a medicament for use in treatment of cancer.

As used herein, and unless otherwise indicated, the terms “treat,” “treating,” and “treatment” contemplate an action that occurs while a patient is suffering from cancer, which reduces the severity of one or more symptoms or effects of cancer, or a related disease or disorder. As used herein, and unless otherwise indicated, the terms “manage,” “managing,” and “management” encompass preventing, delaying, or reducing the severity of a recurrence of cancer in a patient who has already suffered from the cancer. The terms encompass modulating the threshold, development, and/or duration of the cancer, or changing the way that a patient responds to the cancer.

As used herein, and unless otherwise specified, a “therapeutically effective amount” of a compound is an amount sufficient to provide any therapeutic benefit in the treatment or management of cancer, or to delay or minimize one or more symptoms associated with cancer. A therapeutically effective amount of a compound means an amount of the compound, alone or in combination with one or more other therapy and/or therapeutic agent, which provides any therapeutic benefit in the treatment or management of cancer, or related diseases or disorders. The term “therapeutically effective amount” can encompass an amount that cures cancer, improves or reduces cancer, reduces or avoids symptoms or causes of cancer, improves overall therapy, or enhances the therapeutic efficacy of another therapeutic agent.

Toxicity and therapeutic efficacy of the described compounds and compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, expressed as the ratio LD50/ED50. Compounds that exhibit large therapeutic indices are preferred. Compounds that exhibit toxic side effects may be used in certain embodiments, however, care should usually be taken to design delivery systems that target such compounds preferentially to the site of affected tissue, in order to minimize potential damage to uninfected cells and, thereby, reduce side effects.

Data obtained from cell culture assays and animal studies can be used in formulating a range of dosages for use in humans. In certain aspects of the present disclosure, the dosages of such compounds lie within a range of circulating concentrations that include the ED50 with little or no toxicity. The dosage may vary within this range depending on the dosage form employed and the route of administration utilized. For any compound used in the disclosed methods, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Plasma levels may be measured, for example, by high performance liquid chromatography.

When therapeutic treatment is contemplated, the appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human studies of efficacy are undertaken, Phase I clinical studies help establish safe doses. Additionally, the bioactive agent may be complexed with a variety of well established compounds or structures that, for instance, enhance the stability of the bioactive agent, or otherwise enhance its pharmacological properties (e.g., increase in vivo half-life, reduce toxicity, etc.).

In certain embodiments of the present disclosure, the effective dose of the composition or dosage unit can be in the range of about 10 mg/kg to about 0.01 mg/kg, about 10 mg/kg to about 0.025 mg/kg, about 10 mg/kg to about 0.05 mg/kg, about 10 mg/kg to about 0.1 mg/kg, about 10 mg/kg to about 0.25 mg/kg, about 10 mg/kg to about 0.5 mg/kg, about 10 mg/kg to about 1 mg/kg, about 10 mg/kg to about 2.5 mg/kg, about 10 mg/kg to about 5 mg/kg, about 5 mg/kg to about 0.01 mg/kg, about 2.5 mg/kg to about 0.01 mg/kg, about 1 mg/kg to about 0.01 mg/kg, about 0.5 mg/kg to about 0.01 mg/kg, about 0.25 mg/kg to about 0.01 mg/kg, about 0.1 mg/kg to about 0.01 mg/kg, about 0.05 mg/kg to about 0.01 mg/kg, about 0.025 mg/kg to about 0.01 mg/kg, about 5 mg/kg to about 0.025 mg/kg, about 2.5 mg/kg to about 0.05 mg/kg, about 1 mg/kg to about 0.1 mg/kg, about 0.5 mg/kg to about 0.25 mg/kg, or about 3 mg/kg to about 0.1 mg/kg, or so. Thus, in particular embodiments, the effective dose of the composition or dosage unit is about 0.01 mg/kg, about 0.025 mg/kg, about 0.05 mg/kg, about 0.075 mg/kg, about 0.1 mg/kg, about 0.25 mg/kg, about 0.5 mg/kg, about 0.75 mg/kg, about 1 mg/kg, about 2.5 mg/kg, about 3 mg/kg, about 5 mg/kg, about 7.5 mg/kg, or about 10 mg/kg, or so.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims.

### Example 1

In The National Surgical Adjuvant Breast and Bowel Project (“NSABP”) clinical trial B31 cohort, the HER2 assays currently used in routine clinical practice to select patients for HER2 targeted therapies (namely IHC and FISH assays) failed to predict the degree of benefit from trastuzumab, and surprisingly, as shown in Table 1, even patients diagnosed with HER2 negative tumors gained the same degree of benefit as those with HER2 positive breast cancer defined by current HER2 assays (IHC and FISH) (Paik, et al., N. Engl. J. Med. 358:1409-1411, 2008). This data underscores the need to develop a new predictive test that can be used to predict the degree of benefit from HER2 targeted therapies in adjuvant setting.

TABLE 1

| | | Treatment (events/total events) | | | | |
|---|---|---|---|---|---|---|
| | Central | | Chemo plus | RR | | Interaction |
| End Point | HER2 Assay | Chemo | Trastuzumab | (95% CI) | p-value | p-value |
| DFS | Positive | 163/875 | 85/804 | 0.47 | <0.001 | 0.47 |
| | | | | (0.37-0.62) | | |
| | Negative | 20/92 | 7/82 | 0.34 | 0.014 | |
| | | | | (0.14-0.80) | | |
| Overall | Positive | 55/875 | 38/804 | 0.66 | 0.047 | 0.08 |
| Survival | | | | (0.43-0.99) | | |
| | Negative | 10/92 | 1/82 | 0.08 | 0.17 | |
| | | | | (0.01-0.64) | | |

In Table 1, the end points were disease-free survival (“DFS”) or overall survival. The central HER2 assay results were defined as negative if they were negative by both fluorescence in situ hybridization (PathVysion™, Vysis) and immunohistochemical analysis (Herceptest™, Dako), and were defined as positive if either test was positive. Chemotherapy denotes 4 cycles of doxorubicin plus cyclophosphamide followed by 4 cycles of paclitaxel. The 95% confidence intervals (“CI”) and p-values were adjusted according to the number of positive nodes and estrogen-receptor status from the univariate Cox proportional-hazards model for each subgroup in the NSABP B-31 trial.

In order to develop a predictive test for the degree of benefit from trastuzumab or other HER2-targeted therapies, whole genome (trasnscriptome) gene expression profiling was performed on formalin fixed paraffin embedded tumor blocks collected from NSABP trial B-31, which tested the value of adding trastuzumab to standard adjuvant chemotherapy in the treatment of stage 2 or stage 3 breast cancer. The B-31 trial was largely enriched for HER2 positive breast cancer (90%), but also included HER2 negative breast cancer (10%).

The available tumor blocks from NSABP B-31 were divided into two randomly selected cohorts of discovery and validation sets. Microarray gene expression profiling was performed using both Agilent and Affymetrix arrays, and formal statistical tests (in Cox proportional hazard models controlling for clinical variables such as estrogen receptor status, tumor size, age, and number of metastatic axillary lymph nodes) were performed to test the interaction between gene expression and trastuzumab benefit. Since HER2 is a known target for trastuzumab, the initial a priori hypothesis was that HER2 (ERBB2) mRNA expression level is a linear predictor of the degree of benefit from trastuzumab, and improves upon the current generation of IHC- or FISH-based HER2 assays as a predictor of the degree of benefit from trastuzumab.

There are two independent oligonucleotide probes that hybridize to HER2 (ERBB2) mRNA in the Agilent microarray and three probes in the Affymetrix microarray. All five probes showed statistically significant interaction with trastuzumab as shown in Table 2, with interaction p-values ranging from 0.0075 to 00036.

TABLE 2

| Microarray Platform | Probe | Interaction p-value |
|---|---|---|
| Agilent | a_24_p284420 | 0.00092 |
| Agilent | a_23_p89249 | 0.00063 |
| Affymetrix | 234354_x_at | 0.0013 |
| Affymetrix | 216836_s_at | 0.00036 |
| Affymetrix | 210930_s_at | 0.0075 |

Based on these findings, a new HER2 mRNA assay was developed using nanostring platform (Geiss, et al., Nat. Biotechnol. 26:317-325, 2008). The test is based on a commercially available technical platform from Nanostring but with custom designed probe sets including a specific set of reference genes (ACTB, RPLP0, H2ASY, SNRP70) to normalize the expression value of HER2 mRNA. This proprietary set of reference genes were selected from data mining of microarray data from NSABP trial B-27.

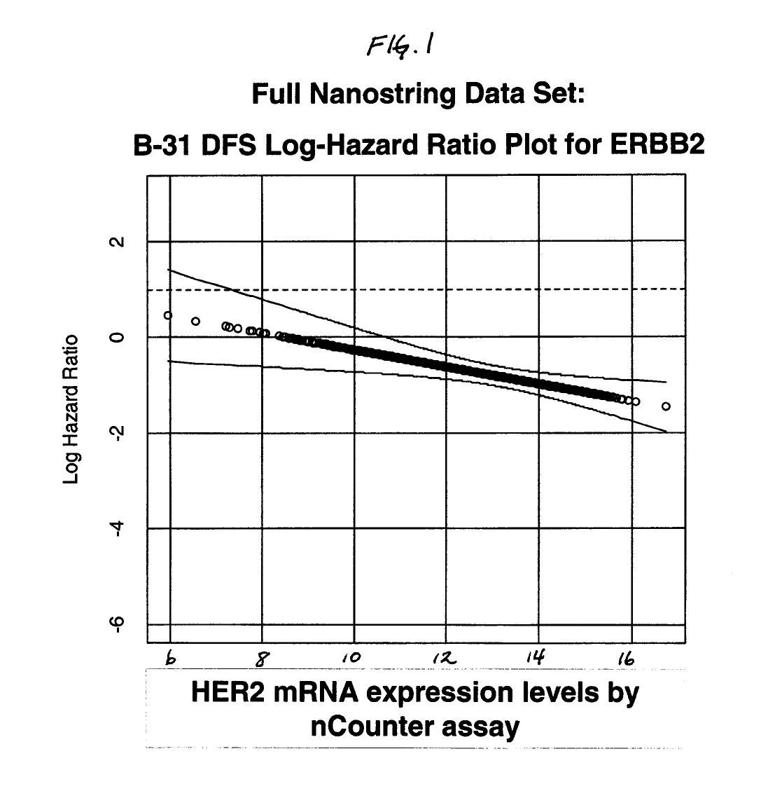

All available tumor blocks from the B-31 trial were examined, and formal statistical tests for interaction between HER2 mRNA and trastuzumab were performed. Nanostring-based HER2 mRNA was strongly predictive of the degree of benefit from trastuzumab in B-31. To illustrate this, log hazard of trastuzumab in B-31 patients is plotted in relation to expression levels of HER2 mRNA (FIG. 1). FIG. 1 shows a linear prediction of the degree of benefit from trastuzumab added to chemotherapy by the level of expression of HER2 mRNA in the treatment of breast cancer. Values above zero on the Y-axis means no benefit, and negative values on the Y-axis mean benefit from trastuzumab. HER2 mRNA levels in FIG. 1 are based on nanostring assays, but other methods of measurement showed similar plots.

With increasing amounts of HER2 mRNA expression in the tumor tissue, there is an increasing degree of benefit from trastuzumab added to chemotherapy in B-31. The cut-off of trastuzumab benefit can be determined from FIG. 1 with confidence intervals. The cut off based on B-31 data is 8.5 normalized HER2 mRNA expression level with a confidence interval of 6 to 10.5.

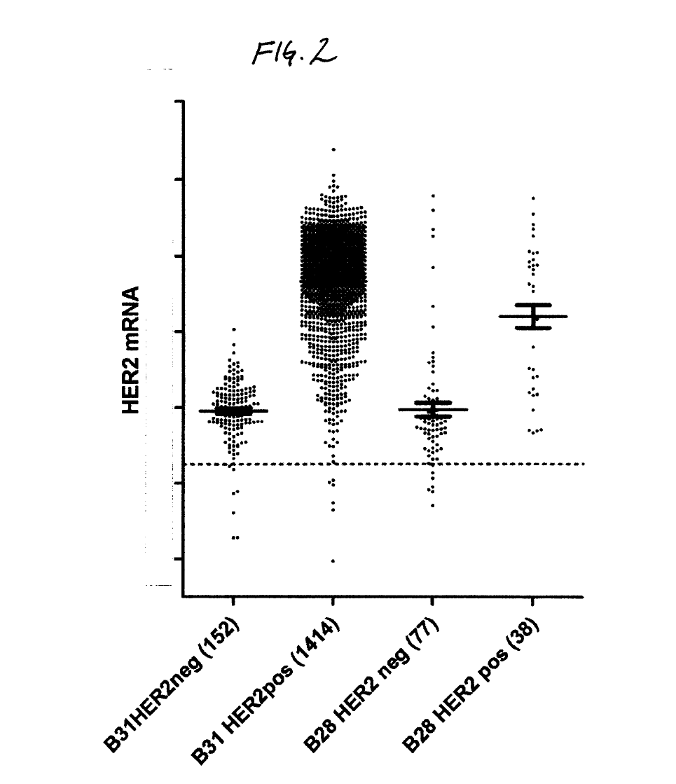

When this cut-off was applied to all breast cancer (B-31 study and B-28 study, which also compares 4 cycles of arimycin (doxorubicin) plus cyclophosphamide versus 4 cycles of AC followed by four cycles of TAXOL® (paclitaxel)), it became evident that a significant proportion of HER2 negative patients would benefit from trastuzumab (FIG. 2). FIG. 2 shows the identification of breast cancer patients who may benefit from trastuzumab in adjuvant setting (stage 2 or stage 3) based on HER2 mRNA measurement. The cut off derived from the nanostring HER2 mRNA assay is applied to a scattergram of tumors that are classified as either HER2 positive or HER2 negative by current clinical HER2 assays (IHC or FISH). The dotted line is the cut-off. It is clear that most breast cancers express HER2 mRNA at levels above the dotted line, suggesting that a significant proportion of patients with breast cancer are expected to benefit from trastuzumab.

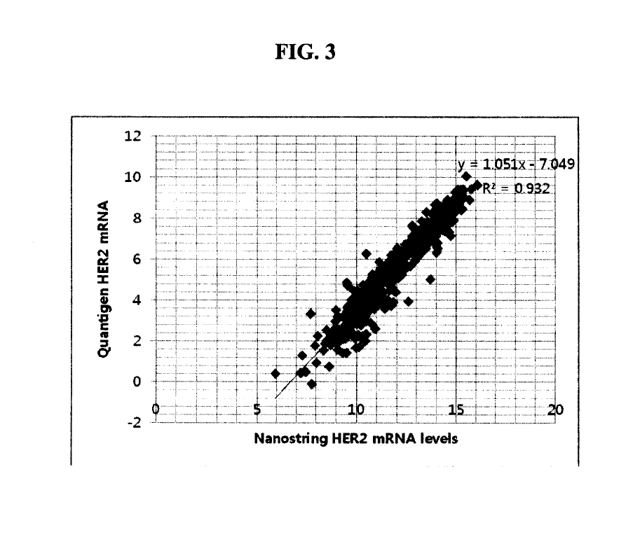

Since HER2 mRNA expression levels linearly correlate with the degree of benefit from trastuzumab, this assay can be utilized to estimate the degree of benefit from trastuzumab before starting the treatment, and this information will help clinicians and patients decide whether to use HER2-targeted therapies, as well as considering other therapies. While the data in this Example is based on HER2 mRNA expression levels measured using either Agilent or Affymetrix arrays, or nanostring platform, the results are applicable broadly to any measure of HER2 mRNA, since a close correlation was demonstrated between HER2 mRNA measured by nanostring and other methods such as Quantigene Plex assay that were performed in a subset of B-31 samples (FIG. 3). FIG. 3 shows the correlation between HER2 mRNA expression levels measured by Nanostring method (nCounter assay) and QuantigenePlex method.

### Example 2

NSABP trial B-31 suggested the efficacy of adjuvant trastuzumab for both HER2-positive and negative breast cancer. In order to develop a predictive model for trastuzumab benefit, gene expression profiling of archived tumor blocks from B-31 was performed. Cases with tumor blocks were randomly divided into a candidate discovery and a confirmation set. A predictive model was built from the candidate discovery cohort (N=588) through gene expression profiling with a custom designed nCounter assay that included candidate predictive and prognostic genes identified from microarray gene expression profiling. Pre-defined cut-points for the predictive model were tested in the confirmation set of 991 patients. Eight predictive genes associated with HER2 (ERBB2, c17orf37, GRB7) or ER (ESR1, NAT1, GATA3, CA12, IGF1R) were selected for the model building. Three dimensional subset treatment effect pattern plot using two principal components of these genes identified a subset with no benefit from trastuzumab, characterized by intermediate-level ERBB2 and high-level ESR1 mRNA expression. In the confirmation set (N=991), the predefined cut-points for this model classified patients into three subsets with differential benefit from trastuzumab with hazard ratios of 1.58 (95% CI: 0.67-3.69, N=100, p=0.29), 0.60 (95% CI: 0.41-0.89, p=0.011, N=449), and 0.28 (95% CI: 0.20-0.41, p<0.0001, N=442). P-value for interaction between the model and trastuzumab was 0.0002. A gene expression based algorithm that predicts the degree of benefit from adjuvant trastuzumab has thus been developed.

Trastuzumab is a monoclonal antibody which is directed against HER2 protein overexpressed in approximately 20% of breast cancer patients with proven efficacy for both macro disease (metastatic and neo-adjuvant setting; Slamon, et al., N. Engl. J. Med. 344:783-792, 2001; Untch, et al., J. Clin. Oncol. 28:2024-2031, 2010) and micro-metastatic disease (adjuvant setting; Piccart-Gebhart, et al., N. Engl. J. Med. 353:1659-1672, 2005; Romond, et al., N. Engl. J. Med. 353:1673-1684, 2005). HER2 positive tumors showed a high rate of pathologic complete response to neo-adjuvant chemotherapy and complete responders tend to have favorable prognosis even without trastuzumab (Carey, et al., Clin. Cancer Res. 13:2329-2334, 2007). In the adjuvant setting, where many patients may have already derived significant benefit from surgery and chemo-endocrine therapy, benefit from addition of trastuzumab could be determined through a complex interaction between HER2 and other confounding variables. In addition, more robust tumor cell response to trastuzumab in adjuvant setting could be expected based on easier trastuzumab access to micro-metastatic tumor cells (Barok, et al., Mol. Cancer. Ther. 6:2065-2072, 2007), less compromised immune system favoring antibody dependent cell mediated cyto-toxicity through trastuzumab (Clynes, et al., Nat. Med. 6:443-446, 2000), and potential dependency of cancer stem cells on HER2 signaling pathway in the absence of HER2 over-expression (Nakanishi, et al., Br. J. Cancer 102:815-826, 2010).

NSABP trial B-31 demonstrated the efficacy of adjuvant trastuzumab added to chemo-endocrine therapy for HER2-positive breast cancer and also suggested a potential efficacy for HER2-negative breast cancer (Romond, et al., supra; Paik, et al., N. Engl. J. Med. 358:1409-1411, 2008). In order to develop a predictive model for the degree of benefit from adjuvant trastuzumab beyond clinical HER2 status, gene expression profiling of archived formalin fixed paraffin embedded tumor blocks (FFPET) from B-31 was performed using nCounter platform (Geiss, et al., Nat. Biotechnol. 26:317-325, 2008). nCounter platform allows multiplexed measurement of gene expression based on direct hybridization without involving enzyme reaction and is suited for profiling degraded RNA extracted from routinely processed FFPET.

Study Design and Patient Cohort

Developing a predictive algorithm using archived FFPET from a finished clinical trial is technically difficult due to degradation of RNA. For predictive model development, the following strategy was used. Among patients who participated in B-31 (N=2043), 1734 patients signed informed consent forms approved by a local Human Investigations Committee in accordance with an assurance filed with and approved by the Department of Health and Human Services to permit use of banked tissue for future studies for cancer and clinical follow up data, available estrogen receptor status, and number of positive nodes. Tumor blocks from 743 patients from the candidate discovery cohort of 800 randomly selected cases were subjected to microarray gene expression profiling to identify candidate predictive genes and prognostic genes, as 57 cases did not yield good RNA amplification product. While biologically relevant genes can be derived using the latter method, previous studies indicated that only about 30% of the genes identified using microarray platform when applied to FFPET could be validated using other technical platforms such as nCounter assay.

Therefore in order to minimize the risk when designing nCounter assay (462 genes) that has a potential to be developed into a clinical assay, not only were genes selected from microarray experiments included, but also other biologically or clinically interesting genes (see below). Since nCounter assay was designed based on follow-up data at the time of unblinding of the trial results, and eventual data analysis was based on 7 year follow up with twice the number of events, many predictive genes were no longer relevant, while other genes that were originally selected based on biology became candidate predictive genes. Because of these circumstances, only nCounter assay results are shown ignoring microarray results.

From the original 743 cases of candidate discovery cohort, after microarray experiments enough RNA was left to perform nCounter assay in 588 cases. Based on analysis of nCounter assay data from 588 cases from the candidate discovery cohort, a single predictive algorithm was committed to and cut-points for each of the categories with varying degrees of expected benefit from trastuzumab. Then these pre-specified cut-points in the remaining 991 cases (confirmation cohort) who were not used for the selection of genes for the predictive algorithm were assessed. There were 57 cases from the discovery cohort that were not subjected to microarray analyses that were included among 991 cases.

nCounter Assay

The nCounter assay was designed with 462 probes to include candidate prognostic and predictive genes from microarray data from the discovery cohort (198 predictive genes and 289 prognostic genes with overlap between the two), 42 prognostic genes from microarray data from NSABP trial B-27 (Bear, et al., J. Clin. Oncol. 24:2019-2027, 2006), PAM 50 genes (Parker, et al., J. Clin. Oncol. 27:1160-1167, 2009), Oncotype Dx genes (Paik, et al., N. Engl. J. Med 351:2817-2826, 2004), and 28 internal reference genes. One hundred nanograms of total RNA were used for the assay. The data for each tumor were normalized for technical variability with the sum of the positive controls inherent to nCounter assay and within sample reference normalized with the geometric mean of 4 internal reference genes (ACTB, RPLP0, SNRP70, H2AFY) which was selected from the microarray data analyses.

Statistical Analysis

Follow-up information was included up to October 2010. Patients from the control arm who crossed over to receive trastuzumab were censored at the time of cross over. The definition of the primary endpoint for this analysis (disease-free survival [DFS]) was previously described (Romond, et al., supra). Gene expression values were categorized into quartiles for screening possible predictive genes since many genes showed non-linearity of their association with treatment effect upon initial review of the data. The gene-by-treatment interaction was tested in the Cox proportional hazard models using the cross-product term of indicator variables for trastuzumab treatment and each marker status with adjustment for nodal status. For single markers other than estrogen receptor, analyses were adjusted for estrogen receptor and nodal status. Correlations between variables were assessed with Spearman's correlation coefficient (r).

The principal component analysis was performed on the final set of selected genes to determine the first two components that would capture most of the variation in the data. Once the two principal components has been chosen, interactions between treatments and the first two principal components (PC1 and PC2) of the candidate predictive genes from nCounter assay were evaluated by the Cox model as well as by means of the non-parametric sub-population treatment effect pattern plot (STEPP; Bonetti and Gelber, Biostatistics 5:465-481, 2004), which is extended for three dimensions (3-D). (See below for detailed methods and code). The 3-D surface plot was drawn with spline interpolation to smooth the plot using S-PLUS ver.8.1 (TIBCO Software Inc., Palo Alto, Calif.). All statistical analyses were done with SAS ver.9.2 (SAS Institute Inc., Cary, N.C.).

STEPP methodology is an exploratory tool for treatment×covariate interaction. Originally, this approach only focused on one covariate, so it was extended for exploring two interaction effects simultaneously because it was believed the treatment effect would be affected by both HER2 associated genes and ER associated genes. For 3-D STEPP analysis, each subsequent subpopulation of 100 patients was formed by removing 50 patients with the lowest Covariate 1 (in this study, PC1) values from the current sub-population and replacing them with the next 50 patients in the ordered list, while fixing 400 sub-population based on the ordered Covariate2 (in this study, PC2) values. Once the moving process based on Covariate 1 values were done, the next subpopulation based on Covariate 2 values were defined by removing 100 patients with the lowest Covariate 2 values from the current subpopulation and replacing them with the next 100 patients in the ordered list. These processes continued until all patients were included in at least one subpopulation. After the overlapping subpopulations were identified, the treatment effect was estimated within each subpopulation using the COX regression models adjusting for nodal status. Furthermore, this calculation was done again exchanging subpopulation setting Covariate 1 for Covariate2 (thus, 400 patients were fixed based on Covariate2 values for consecutive 100 patients subpopulations based on Covariate2 values.) 3-D STEPP analysis results are then shown graphically. All computational processes are provided as an SAS macro program.

The SAS TDSTEPPplot Macro

% TDSTEPPplot is a SAS macro that visually examines the interaction effect of two continuous variables and treatment on failure time with 3D plots, applying COX proportional hazard model. This method is an extension of STEPP analysis, which was originally proposed by Bonetti and Gelber (Stat. Med. 19:2595-2609, 2000).

Invocation and Details

In order to run this macro, the following may need to be included in the SAS program where the file 3dstepp.sas is saved such as: % include “c: \program file\mysasfiles\tdsteppmacro.sas”. Then execute the macro TDSTEPPplot. An example macro call is: options nonotes; % TDSTEPPplot(ds=data1, var1=var1, var2=var2, outds=outsm, rr1=300, rr2=400, r1=50, r2=100, cov=age, trt=treatment, time=surv, cens=censor, cind=1, maxhr=1.5); quit; options notes.

Definition of Macro Variables:

<Parameters for the dataset> DSN: name of the SAS data set containing survival times, status, and covariates.

<Parameters for the variables> Var1: continuous variable name of interest; Var2: another continuous variable name of interest time: survival time; cens: event status indicator variable; icens: censoring status indicator variable value (ex. 1); COVS: list of covariates, separated by blanks. Covariates must be continuous or dummy variables.

<Parameters for STEPP analysis> Rr1: the largest number of subjects in common among consecutive subpopulations for variable 1. Rr2: the number of subjects in each subpopulation for variable 1. (rr2>rr1). R1: the largest number of subjects in common among consecutive subpopulations for variable 2. R2: the number of subjects in each subpopulation for variable 2. (r2>r1)

<Parameters for the outputs> Outds: name of the SAS dataset to create a new output dataset for 3D plot. Maxhr: maximum value of Hazard ratio (Z axis) for the 3-D plot.

The Macro Program is shown in Table 3.

TABLE 3
%macro stepp(r1=, r2=, ds=, var=, cov=, trt=, time=, cens=, cind= );
%let window=%eval(&r2−&r1);
proc means data=&ds;

var &var;
output out=outds n=n;

run;
data outds;set outds;

k=int(n/&window);
call symput(“k”,trim(put(k,best.)));
call symput(“obsn”,trim(put(n,best.)));

run;
proc rank data=&ds out=&ds;

var &var;
ranks rank;

run;
%do i=1 %to &k;

%let f=%eval(1+&window*(&i.−1));
%let l=%eval(&f+&r2);
%if &i<&k %then %do;

data data&i; set &ds;

if &f=< rank<=&l;

%end;
run;

%if &i=&k %then %do;

data data&i; set &ds;

if &f=< rank;

%end;
run;

proc means data=data&i;

var &var;
output out=out&i median=med;

run;
data out&i; set out&i;

call symput(“median”,trim(put(med,best.)));

run;
proc phreg data=data&i;

model &time*&cens(&cind)=&TRT &cov /rl;
Hazardratio &TRT;
ods output HazardRatios =hr&i;

run;

data hr&i; set
hr&i; i=&i;
median=&median;

run;

%end;
data hr&var; set %do s=1 %to &k; hr&s %end;; run;
%mend;

%macro TDSTEPP(ds=, var2=, var1=, rr1=, rr2=, r1=, r2=, cov=,
trt=, time=, cens=, cind= );

data &ds;set &ds; drop rank:; run;
%let window1=%eval(&rr2−&rr1);
proc means data=&ds;

var &var1;
output out=outds1 n=n;

run;
data outds1;set outds1;

kk=int(n/&window1);
call symput(“kk”,trim(put(kk,best.)));
call symput(“nall”,trim(put(n,best.)));

run;
proc rank data=&ds out=&ds;

var &var1;
ranks rank1;

run;
%do q=1 %to &kk;

%let f1=%eval(1+&window1.*(&q.−1));
%let l1=%eval(&f1+&rr2.);
%if &q<&kk %then %do;

data d&q; set &ds; if &f1=< rank1<=&l1; run;

%end;
%if &q=&kk %then %do;

data d&q; set &ds; if &f1=< rank1; run;

%end;
proc means data=d&q;

var &var1;
output out=out1_&q median=med;

run;
data out1_&q; set out1_&q;

call symput(“median1”,trim(put(med,best.)));

run;

%stepp(r1=&r1, r2=&r2, ds=d&q, var=&var2, cov=&cov, trt=&trt,
time=&time, cens=&cens, cind=&cind );

data hrr&q; set hr&var2;

q=&q;
&var1=&median1;
rename median=&var2;

run;

%end;
data hrall&var1; set %do t=1 %to &kk; hrr&t %end;; run;
%mend;

%macro TDSTEPPplot(ds=, var1=, var2=, outds=, rr1=, rr2=,
r1=, r2=, cov=, trt=, time=, cens=, cind= , maxhr= );

ods listing close;
%TDSTEPP(ds=&ds, var2=&var2, var1=&var1, rr1=&rr1, rr2=&rr2, r1=&r1, r2=&r2,

cov=&cov, trt=&trt, time=&time, cens=&cens, cind=&cind );

quit;

%TDSTEPP(ds=&ds, var2=&var1, var1=&var2, rr1=&rr1, rr2=&rr2,
r1=&r1, r2=&r2, cov=&cov, trt=&trt, time=&time, cens=&cens,
cind=&cind );

ods listing;
data hrall; set hrall&var1 hrall&var2;run;
proc means data=hrall;

var &var1;
output out=out1 max=max1 min=min1;

run;
data out1;

set out1;
call symput(“max1”,trim(put(max1,best.)));
call symput(“min1”,trim(put(min1,best.)));

run;
proc means data=hrall;

var &var2;
output out=out2 max=max2 min=min2;

run;
data out2; set out2;

call symput(“max2”,trim(put(max2,best.)));

call symput(“min2”,trim(put(min2,best.)));
run;
proc g3grid data=hrall out=&outds;

grid &var1*&var2=HazardRatio / spline smooth=.2

axis1=&min1. to &max1. by 0.5
axis2=&min2. to &max2. by 0.5;

run;
goptions reset=all border ;
axis3 order=(0 to &maxhr by 0.1) label=none;
proc g3d data=&outds;

plot &var1*&var2-HazardRatio / rotate=60 grid zaxis=axis3 zticknum=14

zmin=0 zmax=1.5;

run;
quit;
%mend;

Results

Results of nCounter Assay in the Candidate Discovery Cohort (N=588) and Development of a Prediction Model

Although microarray gene expression analyses of 743 tumors from the discovery cohort were performed, the genes discovered from the microarray experiments could only be partially technically validated using other platforms such as nCounter assay. Therefore other biologically and clinically relevant genes were included in the design of the nCounter assay. nCounter assay is ideal for multiplexed quantification of relative gene expression levels using RNA extracted from FFPET samples since it uses short hybridization sequences and does not depend on enzymatic reaction.

In order to develop a predictive algorithm, it was first tried to identify reproducibly predictive genes by performing ten-fold jack-knifing process. The results of statistical tests for gene-by-trastuzumab interaction terms in Cox models adjusting for the number of positive nodes are shown in Table 4.

TABLE 4

| Number | | | | |
|---|---|---|---|---|
| of times | | | | |
| significant | mean | maximum | minimum | |
| during | p-value | p-value | p-value | |
| 1-fold Jack | from 10- | from 10 | from 10 | |
| Knifing | fold jack | fold jack | fold jack | |
| process | knifing | knifing | knifing | gene symbol |
| 10 | 0.0025 | 0.0054 | 0.0002 | FLOT2 |
| 10 | 0.0049 | 0.01 | 0.0008 | UNC119 |
| 10 | 0.0051 | 0.0136 | 0.0008 | TUBB2C |
| 10 | 0.0054 | 0.0131 | 0.0016 | XYLT1 |
| 10 | 0.0057 | 0.0151 | 0.0018 | SLC39A14 |
| 10 | 0.0059 | 0.0269 | 0.0007 | CA12 |
| 10 | 0.007 | 0.0154 | 0.001 | GATA3 |
| 9 | 0.0078 | 0.0509 | 0.0003 | GTF3C2 |
| 10 | 0.0088 | 0.0223 | 0.0014 | SLC39A14 |
| 10 | 0.0095 | 0.025 | 0.0013 | CA12 |
| 10 | 0.0145 | 0.0347 | 0.0024 | FTH1 |
| 10 | 0.0155 | 0.0385 | 0.0013 | SUPT6H |
| 10 | 0.0156 | 0.0349 | 0.0041 | ACVR1B |
| 9 | 0.0166 | 0.0533 | 0.005 | DKFZP434A0131 |
| 10 | 0.0181 | 0.0357 | 0.0014 | RPL23A |
| 9 | 0.0188 | 0.0825 | 0.0012 | ILF2 |
| 9 | 0.0194 | 0.0591 | 0.0056 | DNAJC4 |
| 10 | 0.02 | 0.0477 | 0.002 | ABHD2 |
| 10 | 0.0214 | 0.0476 | 0.0093 | ZACN |
| 9 | 0.0239 | 0.0976 | 0.0041 | TPBG |
| 9 | 0.0241 | 0.053 | 0.0052 | DNAJC4 |
| 10 | 0.0242 | 0.0396 | 0.0034 | FAM84B |
| 9 | 0.0243 | 0.0562 | 0.0042 | SPDEF |
| 8 | 0.0277 | 0.0808 | 0.0074 | DAD1 |
| 8 | 0.0297 | 0.1148 | 0.0039 | CASC3 |
| 9 | 0.03 | 0.0535 | 0.0044 | MYADM |
| 9 | 0.0316 | 0.1292 | 0.0079 | PTTG1 |
| 8 | 0.0329 | 0.0827 | 0.0059 | UHMK1 |
| 6 | 0.0346 | 0.0666 | 0.0059 | TMBIM6 |
| 8 | 0.0348 | 0.0911 | 0.006 | THOP1 |
| 9 | 0.0364 | 0.0863 | 0.0058 | ANGPTL2 |
| 8 | 0.0366 | 0.139 | 0.005 | ISOC1 |
| 9 | 0.0379 | 0.086 | 0.0131 | TMSB10 |
| 9 | 0.0388 | 0.2252 | 0.0056 | PIK3CA |
| 7 | 0.0401 | 0.107 | 0.0097 | SLC7A2 |
| 6 | 0.0407 | 0.1022 | 0.0088 | ORC6L |
| 6 | 0.0408 | 0.0607 | 0.0116 | SPP1 |
| 6 | 0.0411 | 0.0881 | 0.0083 | CD9 |
| 7 | 0.0426 | 0.095 | 0.009 | PCK2 |
| 7 | 0.0433 | 0.097 | 0.0125 | CEACAM1 |
| 6 | 0.0437 | 0.0896 | 0.0159 | RPL21 |
| 7 | 0.0442 | 0.1008 | 0.0084 | C17orf37 |
| 7 | 0.0458 | 0.1119 | 0.016 | KHSRP |
| 7 | 0.0462 | 0.1588 | 0.0111 | RASSF7 |
| 5 | 0.0466 | 0.073 | 0.0196 | RPL21 |
| 7 | 0.0477 | 0.1475 | 0.0127 | RPL34 |
| 6 | 0.0485 | 0.1114 | 0.0064 | ERBB2 |
| 6 | 0.0489 | 0.1281 | 0.0116 | RPL23A |
| 6 | 0.0497 | 0.1363 | 0.0083 | NUF2 |
| 5 | 0.0516 | 0.0997 | 0.0122 | EGFR |
| 6 | 0.0525 | 0.1375 | 0.0126 | ENPP1 |
| 7 | 0.0528 | 0.0949 | 0.0138 | ZNF609 |
| 6 | 0.0542 | 0.1148 | 0.007 | NLK |
| 6 | 0.0574 | 0.1421 | 0.0096 | ERBB2 |
| 3 | 0.0593 | 0.0954 | 0.0112 | IGF1R |
| 8 | 0.0603 | 0.2704 | 0.0089 | L3MBTL2 |
| 5 | 0.0612 | 0.1314 | 0.0336 | LOXL3 |
| 5 | 0.0617 | 0.1868 | 0.0046 | TPBG |
| 6 | 0.0623 | 0.1546 | 0.0151 | ACVR1B |
| 4 | 0.0631 | 0.1314 | 0.0217 | PTP4A2 |
| 3 | 0.0636 | 0.116 | 0.0202 | GATA3 |
| 6 | 0.0648 | 0.1645 | 0.0038 | PRR3 |
| 5 | 0.0656 | 0.2032 | 0.0131 | SLC39A14 |
| 4 | 0.0657 | 0.1155 | 0.0106 | C9orf58 |
| 5 | 0.0665 | 0.1483 | 0.026 | B4GALT1 |
| 6 | 0.0676 | 0.2062 | 0.0203 | TBX21 |
| 5 | 0.0682 | 0.1752 | 0.014 | FBXW11 |
| 5 | 0.0687 | 0.1844 | 0.0097 | MTCH2 |
| 4 | 0.0701 | 0.2389 | 0.0297 | ZNF124 |
| 4 | 0.0705 | 0.151 | 0.0154 | XYLT1 |
| 5 | 0.0714 | 0.1418 | 0.024 | KRT7 |
| 3 | 0.079 | 0.1425 | 0.0191 | PADI2 |
| 4 | 0.0797 | 0.174 | 0.0259 | CA12 |
| 2 | 0.0875 | 0.162 | 0.0256 | KRT7 |
| 4 | 0.088 | 0.2208 | 0.0259 | PTP4A2 |
| 3 | 0.0889 | 0.3082 | 0.0289 | EHMT1 |
| 1 | 0.0908 | 0.1753 | 0.0179 | ANGPTL4 |
| 3 | 0.0912 | 0.2083 | 0.0217 | LASS6 |
| 1 | 0.0914 | 0.1663 | 0.0157 | IGKV1-5 |
| 3 | 0.0914 | 0.1889 | 0.0359 | MTCH2 |
| 2 | 0.0925 | 0.1579 | 0.0336 | KIF2C |
| 4 | 0.0926 | 0.233 | 0.024 | ASPHD2 |
| 4 | 0.0949 | 0.2731 | 0.0235 | KLHL25 |
| 4 | 0.0952 | 0.2981 | 0.0222 | GRB7 |
| 2 | 0.0952 | 0.23 | 0.0335 | MED13L |
| 4 | 0.096 | 0.1809 | 0.0187 | FAM127A |
| 4 | 0.0966 | 0.2073 | 0.0305 | FAM148A |
| 2 | 0.0975 | 0.2084 | 0.011 | MYB |
| 2 | 0.0978 | 0.2047 | 0.03 | SNX5 |
| 3 | 0.0987 | 0.2458 | 0.0268 | ZC3H15 |
| 7 | 0.0993 | 0.3792 | 0.0168 | ELN |
| 2 | 0.1 | 0.3233 | 0.0221 | PTP4A2 |
| 1 | 0.1013 | 0.1945 | 0.0263 | MYADM |
| 3 | 0.1027 | 0.2017 | 0.037 | C1orf93 |
| 2 | 0.1038 | 0.2977 | 0.0315 | B4GALT1 |
| 3 | 0.1039 | 0.2121 | 0.023 | ESR1 |
| 3 | 0.1044 | 0.2061 | 0.027 | UBE2W |
| 3 | 0.1052 | 0.3319 | 0.0388 | UBE2C |
| 0 | 0.1056 | 0.1868 | 0.0637 | SOX4 |
| 4 | 0.1065 | 0.3086 | 0.0148 | LOC442270 |
| 4 | 0.1066 | 0.3086 | 0.0158 | TMBIM6 |
| 4 | 0.1075 | 0.2562 | 0.0207 | PGRMC2 |
| 2 | 0.1077 | 0.2182 | 0.0332 | IGF1R |
| 2 | 0.1077 | 0.2508 | 0.0213 | SSBP2 |
| 3 | 0.1079 | 0.2653 | 0.0374 | ZC3HAV1L |
| 2 | 0.1085 | 0.2962 | 0.0098 | MGC70870 |
| 1 | 0.1094 | 0.212 | 0.0432 | MYADM |
| 5 | 0.1117 | 0.3441 | 0.0161 | TMBIM6 |
| 1 | 0.1118 | 0.2436 | 0.0399 | ACAD9 |
| 2 | 0.1126 | 0.3157 | 0.0165 | ESR1 |
| 0 | 0.1147 | 0.3465 | 0.0518 | NDC80 |
| 3 | 0.1148 | 0.2837 | 0.0251 | KCNE1 |
| 0 | 0.115 | 0.2392 | 0.0533 | THOP1 |
| 4 | 0.1153 | 0.3512 | 0.0352 | ABHD2 |
| 3 | 0.1153 | 0.4057 | 0.0161 | MGC70870 |
| 1 | 0.1164 | 0.2129 | 0.0337 | RPL21 |
| 1 | 0.1164 | 0.4675 | 0.03 | CLIC1 |
| 1 | 0.1168 | 0.2156 | 0.0485 | TMBIM1 |
| 1 | 0.1188 | 0.2397 | 0.0306 | MIA |
| 4 | 0.119 | 0.3063 | 0.0135 | PSMD3 |
| 2 | 0.1225 | 0.3202 | 0.0164 | KLHL25 |
| 4 | 0.1228 | 0.3382 | 0.0353 | AURKA |
| 2 | 0.1236 | 0.2737 | 0.0325 | KRT18 |
| 1 | 0.124 | 0.2089 | 0.0441 | POLR2L |
| 1 | 0.1247 | 0.2142 | 0.0416 | BLVRA |
| 2 | 0.1259 | 0.2619 | 0.0266 | PRPF40A |
| 1 | 0.1264 | 0.2285 | 0.0147 | TBXAS1 |
| 3 | 0.1268 | 0.4372 | 0.0173 | KCNE1 |
| 1 | 0.127 | 0.4015 | 0.0486 | LSM14A |
| 2 | 0.1273 | 0.3058 | 0.0308 | FURIN |
| 1 | 0.1275 | 0.2569 | 0.0403 | ADFP |
| 4 | 0.1278 | 0.3995 | 0.0283 | L3MBTL2 |
| 2 | 0.1286 | 0.4374 | 0.0383 | FOXA1 |
| 1 | 0.1289 | 0.4577 | 0.0392 | LOC442270 |
| 2 | 0.1301 | 0.2629 | 0.0402 | Kua-UEV |
| 1 | 0.1301 | 0.2505 | 0.0402 | TBX10 |
| 1 | 0.1306 | 0.2214 | 0.0389 | SREBF2 |
| 0 | 0.1318 | 0.2826 | 0.061 | C17orf37 |
| 1 | 0.1321 | 0.2319 | 0.0381 | UBTD1 |
| 3 | 0.1326 | 0.3082 | 0.0319 | NAT1 |
| 1 | 0.1326 | 0.2468 | 0.0374 | RPL34 |
| 0 | 0.1326 | 0.2183 | 0.0542 | UHMK1 |
| 1 | 0.133 | 0.2916 | 0.0287 | SPP1 |
| 0 | 0.1335 | 0.3095 | 0.0751 | RBM14 |
| 0 | 0.1336 | 0.311 | 0.0622 | HSPBP1 |
| 1 | 0.1336 | 0.2821 | 0.0335 | TYMS |
| 0 | 0.1341 | 0.2537 | 0.0501 | ANLN |
| 1 | 0.1346 | 0.2968 | 0.0488 | KRT81 |
| 2 | 0.1349 | 0.2318 | 0.0339 | CUGBP1 |
| 1 | 0.1351 | 0.2631 | 0.0451 | PPP2R2D |
| 1 | 0.1354 | 0.2027 | 0.0454 | BBC3 |
| 1 | 0.1356 | 0.207 | 0.0375 | KRT81 |
| 1 | 0.1363 | 0.3257 | 0.0262 | LOXL3 |
| 3 | 0.1383 | 0.3505 | 0.0219 | ORMDL3 |
| 3 | 0.1385 | 0.4207 | 0.0208 | CCL21 |
| 0 | 0.1391 | 0.2146 | 0.0549 | HSPBP1 |
| 0 | 0.1391 | 0.3087 | 0.0597 | LOXL3 |
| 1 | 0.1397 | 0.2978 | 0.0203 | FKBP3 |
| 0 | 0.1402 | 0.2267 | 0.0605 | UGCG |
| 1 | 0.1405 | 0.2634 | 0.0396 | MYB |
| 1 | 0.1405 | 0.3013 | 0.0494 | ORC6L |
| 0 | 0.1428 | 0.2423 | 0.0658 | POLR2L |
| 1 | 0.1432 | 0.2928 | 0.0424 | FBXO15 |
| 0 | 0.1435 | 0.2405 | 0.0832 | CRTC2 |
| 1 | 0.1441 | 0.4018 | 0.0405 | TBX10 |
| 2 | 0.1449 | 0.2684 | 0.0376 | GUSBL2 |
| 0 | 0.1452 | 0.3304 | 0.0655 | UNC119 |
| 1 | 0.1452 | 0.321 | 0.045 | CYBRD1 |
| 0 | 0.1476 | 0.3627 | 0.0568 | PTTG1 |
| 1 | 0.1477 | 0.3087 | 0.0474 | ESR1 |
| 1 | 0.1504 | 0.3412 | 0.0392 | ACVR1B |
| 0 | 0.1512 | 0.2638 | 0.054 | TYMS |
| 2 | 0.1519 | 0.2818 | 0.0492 | FURIN |
| 0 | 0.1519 | 0.3033 | 0.0643 | POM121L9P |
| 1 | 0.1546 | 0.3953 | 0.0426 | FTH1 |
| 1 | 0.1568 | 0.2943 | 0.0162 | FBXO15 |
| 0 | 0.1587 | 0.3133 | 0.0543 | CFLP1 |
| 1 | 0.1588 | 0.2832 | 0.027 | SFRP1 |
| 1 | 0.1597 | 0.2758 | 0.0486 | FLJ22795 |
| 1 | 0.1612 | 0.4295 | 0.0377 | PIK3CA |
| 1 | 0.1631 | 0.3609 | 0.049 | CIAPIN1 |
| 0 | 0.1642 | 0.2968 | 0.0794 | URM1 |
| 0 | 0.1648 | 0.2875 | 0.0641 | NEBL |
| 2 | 0.1654 | 0.3332 | 0.0344 | PGR |
| 0 | 0.1665 | 0.3038 | 0.0658 | SPTAN1 |
| 1 | 0.1668 | 0.5187 | 0.0318 | TPBG |
| 1 | 0.1671 | 0.4113 | 0.0406 | BRAF |
| 0 | 0.1676 | 0.2907 | 0.0648 | CCDC24 |
| 1 | 0.1679 | 0.2974 | 0.0463 | SNHG5 |
| 2 | 0.1699 | 0.3429 | 0.0247 | REPS2 |
| 0 | 0.1704 | 0.335 | 0.0583 | FURIN |
| 1 | 0.1704 | 0.3224 | 0.0487 | AKT1 |
| 1 | 0.1708 | 0.3422 | 0.0481 | ANGPTL2 |
| 0 | 0.1708 | 0.3428 | 0.0518 | KIAA0652 |
| 1 | 0.1728 | 0.263 | 0.0425 | PSMD3 |
| 0 | 0.1733 | 0.2574 | 0.0904 | TAPBP |
| 1 | 0.1737 | 0.2782 | 0.0484 | HERC2P4 |
| 1 | 0.1737 | 0.5989 | 0.0482 | C16orf14 |
| 0 | 0.1747 | 0.4094 | 0.076 | SLC7A2 |
| 1 | 0.1748 | 0.2931 | 0.0435 | ABCF2 |
| 1 | 0.1754 | 0.3594 | 0.0213 | NAT1 |
| 1 | 0.1754 | 0.3153 | 0.0395 | SSBP2 |
| 0 | 0.1771 | 0.4647 | 0.0501 | CIAPIN1 |
| 0 | 0.1782 | 0.3452 | 0.0946 | ILF2 |
| 0 | 0.1785 | 0.3608 | 0.0722 | TMEM174 |
| 1 | 0.1808 | 0.5008 | 0.0497 | NECAB3 |
| 0 | 0.1819 | 0.3369 | 0.0685 | YWHAZ |
| 2 | 0.1823 | 0.3831 | 0.0104 | CD9 |
| 1 | 0.1828 | 0.3374 | 0.0272 | LCE3E |
| 0 | 0.1834 | 0.2813 | 0.0974 | THSD4 |
| 0 | 0.1844 | 0.245 | 0.1379 | ACTB |
| 1 | 0.1848 | 0.2965 | 0.0409 | IGHV1-69 |
| 0 | 0.1863 | 0.4299 | 0.073 | C20orf144 |
| 0 | 0.1869 | 0.4663 | 0.0743 | PIK3CA |
| 1 | 0.1873 | 0.3484 | 0.033 | NAT1 |
| 1 | 0.1879 | 0.3093 | 0.0366 | CTSL2 |
| 0 | 0.1881 | 0.3848 | 0.0554 | GTF3C2 |
| 0 | 0.1882 | 0.3169 | 0.0591 | TFRC |
| 0 | 0.1884 | 0.3586 | 0.0856 | KRT81 |
| 0 | 0.1892 | 0.3056 | 0.0958 | REPS2 |
| 0 | 0.1895 | 0.378 | 0.0625 | GRB7 |
| 0 | 0.1917 | 0.3347 | 0.068 | ATAD3A |
| 1 | 0.192 | 0.3626 | 0.0271 | HPS6 |
| 0 | 0.1923 | 0.3721 | 0.0738 | CEP55 |
| 0 | 0.1934 | 0.4 | 0.0528 | GTF3C2 |
| 0 | 0.194 | 0.4355 | 0.0746 | GCGR |
| 0 | 0.1949 | 0.5025 | 0.052 | CD9 |
| 0 | 0.1954 | 0.4391 | 0.0603 | ZFP36L1 |
| 0 | 0.1958 | 0.3983 | 0.1003 | IGH@ |
| 0 | 0.196 | 0.552 | 0.0925 | ZFP36L1 |
| 1 | 0.1962 | 0.3786 | 0.0328 | hCG_1642354 |
| 0 | 0.1971 | 0.5528 | 0.0552 | CXorf56 |
| 0 | 0.1982 | 0.2783 | 0.0534 | CASC3 |
| 0 | 0.1986 | 0.3211 | 0.0519 | UBE2N |
| 0 | 0.1999 | 0.4872 | 0.0861 | DKFZP434A0131 |
| 0 | 0.2 | 0.5259 | 0.0536 | MAPT |
| 0 | 0.2013 | 0.4253 | 0.0667 | IL6ST |
| 0 | 0.2024 | 0.2755 | 0.1174 | ASPHD2 |
| 0 | 0.2028 | 0.4818 | 0.0768 | GCGR |
| 0 | 0.2033 | 0.4887 | 0.0667 | KRTAP6-3 |
| 0 | 0.2039 | 0.4344 | 0.1259 | PPIA |
| 1 | 0.2051 | 0.6008 | 0.0316 | HRH2 |
| 0 | 0.2052 | 0.4033 | 0.0771 | SFRP1 |
| 0 | 0.2056 | 0.3406 | 0.0645 | POLDIP2 |
| 0 | 0.2064 | 0.3995 | 0.0858 | IDUA |
| 0 | 0.2074 | 0.4174 | 0.0627 | MELK |
| 0 | 0.209 | 0.5488 | 0.0754 | LAYN |
| 0 | 0.209 | 0.4421 | 0.1147 | ZC3H15 |
| 0 | 0.2108 | 0.2828 | 0.1321 | SIAH2 |
| 0 | 0.2109 | 0.3212 | 0.0948 | PADI2 |
| 1 | 0.2117 | 0.5989 | 0.0417 | RAB27B |
| 0 | 0.2129 | 0.4565 | 0.1031 | ENO1 |
| 0 | 0.2136 | 0.3619 | 0.0562 | CD24 |
| 0 | 0.2147 | 0.3855 | 0.099 | SLC25A5 |
| 0 | 0.2155 | 0.4141 | 0.0593 | CLIC1 |
| 0 | 0.2161 | 0.3202 | 0.1025 | RAB27B |
| 1 | 0.2161 | 0.4247 | 0.0206 | CCL21 |
| 0 | 0.2179 | 0.3817 | 0.1189 | MYB |
| 0 | 0.2182 | 0.3441 | 0.0967 | C1orf212 |
| 0 | 0.22 | 0.4717 | 0.0563 | MRPS36 |
| 0 | 0.22 | 0.4899 | 0.1048 | PCBD2 |
| 0 | 0.2217 | 0.405 | 0.1032 | KRT14 |
| 0 | 0.2227 | 0.3292 | 0.1048 | THSD4 |
| 0 | 0.224 | 0.4659 | 0.0945 | UGCG |
| 0 | 0.2244 | 0.4271 | 0.0562 | ARL17 |
| 0 | 0.225 | 0.4044 | 0.0935 | CSNK1D |
| 0 | 0.2251 | 0.45 | 0.0864 | GSTM1 |
| 0 | 0.2251 | 0.5311 | 0.0705 | RPLP0 |
| 0 | 0.2257 | 0.5385 | 0.0566 | KRTAP6-3 |
| 0 | 0.2268 | 0.4222 | 0.0921 | PHACTR4 |
| 0 | 0.2281 | 0.5222 | 0.0595 | C20orf67 |
| 0 | 0.229 | 0.6194 | 0.1066 | PSMC5 |
| 0 | 0.2295 | 0.4129 | 0.0914 | PCTK2 |
| 0 | 0.23 | 0.4047 | 0.06 | hCG_1642354 |
| 0 | 0.2303 | 0.423 | 0.0704 | HIBCH |
| 0 | 0.2308 | 0.4319 | 0.079 | CHD6 |
| 1 | 0.2326 | 0.4362 | 0.0331 | CASC3 |
| 0 | 0.233 | 0.3518 | 0.1259 | ANLN |
| 0 | 0.233 | 0.5514 | 0.0531 | LOC730275 |
| 0 | 0.2331 | 0.4251 | 0.1088 | ETS2 |
| 0 | 0.2331 | 0.3735 | 0.1297 | IMPAD1 |
| 1 | 0.2335 | 0.3654 | 0.0476 | RASSF7 |
| 1 | 0.2335 | 0.4652 | 0.0481 | RPS28 |
| 0 | 0.2352 | 0.7334 | 0.0923 | SMCP |
| 0 | 0.2353 | 0.4502 | 0.1493 | AK1 |
| 1 | 0.2355 | 0.3806 | 0.0327 | POLR3H |
| 0 | 0.237 | 0.3771 | 0.122 | NME3 |
| 0 | 0.2371 | 0.3776 | 0.1038 | FBXW11 |
| 1 | 0.2372 | 0.5584 | 0.0377 | HIST1H2AA |
| 0 | 0.2376 | 0.3633 | 0.0901 | BIRC5 |
| 0 | 0.2377 | 0.4972 | 0.0864 | SLC39A6 |
| 0 | 0.2386 | 0.5099 | 0.0784 | POLD4 |
| 1 | 0.2393 | 0.4379 | 0.0434 | TRIB3 |
| 0 | 0.2401 | 0.3576 | 0.1003 | C9orf58 |
| 0 | 0.2402 | 0.5139 | 0.0824 | TMSB10 |
| 0 | 0.2405 | 0.576 | 0.0778 | NEBL |
| 0 | 0.2405 | 0.6161 | 0.0885 | ST6GALNAC4 |
| 0 | 0.241 | 0.4734 | 0.0764 | POLDIP2 |
| 0 | 0.2434 | 0.4973 | 0.0992 | BLVRA |
| 1 | 0.2439 | 0.5358 | 0.0238 | HPS6 |
| 0 | 0.244 | 0.5325 | 0.0631 | RAF1 |
| 0 | 0.2456 | 0.5851 | 0.0711 | GGA2 |
| 0 | 0.2464 | 0.4561 | 0.1281 | SREBF2 |
| 0 | 0.2467 | 0.516 | 0.1311 | PITPNC1 |
| 0 | 0.2475 | 0.5387 | 0.0732 | LOC346887 |
| 0 | 0.2476 | 0.5254 | 0.063 | CCNB1 |
| 0 | 0.2481 | 0.4521 | 0.1128 | SIAH2 |
| 0 | 0.2488 | 0.4233 | 0.1136 | C8orf73 |
| 0 | 0.2499 | 0.4664 | 0.0668 | IL6ST |
| 0 | 0.2503 | 0.608 | 0.0958 | ABHD2 |
| 0 | 0.2506 | 0.4927 | 0.0903 | BAG1 |
| 0 | 0.251 | 0.4168 | 0.075 | LOC346887 |
| 1 | 0.2517 | 0.6055 | 0.0282 | KRTAP6-3 |
| 0 | 0.2521 | 0.5378 | 0.0596 | HN1 |
| 1 | 0.2528 | 0.4936 | 0.0453 | ADAMTSL5 |
| 0 | 0.2529 | 0.6362 | 0.0702 | HIST2H2BE |
| 0 | 0.2531 | 0.5166 | 0.1086 | BRAF |
| 0 | 0.2539 | 0.7009 | 0.0587 | FOXA1 |
| 0 | 0.2556 | 0.5933 | 0.1421 | FLJ35390 |
| 0 | 0.2557 | 0.5763 | 0.0907 | GUSB |
| 1 | 0.2589 | 0.5904 | 0.0293 | CTSL2 |
| 1 | 0.2595 | 0.488 | 0.0483 | HERC2P4 |
| 0 | 0.2615 | 0.4635 | 0.1195 | ZNF124 |
| 0 | 0.2629 | 0.4835 | 0.0752 | EPN2 |
| 0 | 0.263 | 0.4065 | 0.145 | CEACAM1 |
| 0 | 0.2641 | 0.5647 | 0.0611 | VPS45A |
| 0 | 0.2642 | 0.523 | 0.0875 | TUBB2C |
| 1 | 0.2653 | 0.6265 | 0.0399 | GPR160 |
| 0 | 0.2654 | 0.591 | 0.0825 | MAPT |
| 0 | 0.2661 | 0.5232 | 0.1261 | BCAS1 |
| 0 | 0.2673 | 0.4542 | 0.1233 | NME3 |
| 0 | 0.2678 | 0.5888 | 0.0602 | MDM2 |
| 1 | 0.2686 | 0.5369 | 0.0419 | THRAP1 |
| 0 | 0.2695 | 0.5733 | 0.1591 | CCDC74A |
| 0 | 0.2713 | 0.6686 | 0.146 | SNHG5 |
| 0 | 0.2718 | 0.5621 | 0.0842 | HDAC4 |
| 0 | 0.272 | 0.5353 | 0.1183 | SF3B3 |
| 0 | 0.2721 | 0.5958 | 0.1201 | C19orf28 |
| 0 | 0.2724 | 0.4892 | 0.1002 | ADFP |
| 0 | 0.2732 | 0.6091 | 0.1586 | HSPA8 |
| 0 | 0.2741 | 0.3818 | 0.1661 | MED13L |
| 0 | 0.2755 | 0.6604 | 0.1028 | ANGPTL4 |
| 1 | 0.277 | 0.8196 | 0.0452 | BTG2 |
| 0 | 0.2799 | 0.7136 | 0.0581 | KCNE1 |
| 0 | 0.2801 | 0.7793 | 0.0869 | LOC100128062 |
| 0 | 0.2808 | 0.429 | 0.1566 | C8orf73 |
| 0 | 0.2809 | 0.4603 | 0.082 | H2AFY |
| 0 | 0.2816 | 0.4557 | 0.0651 | AK1 |
| 0 | 0.282 | 0.6567 | 0.0946 | HERC2P4 |
| 0 | 0.2823 | 0.4587 | 0.1823 | MYBL2 |
| 0 | 0.2823 | 0.556 | 0.1192 | BCL2 |
| 0 | 0.2828 | 0.4205 | 0.1837 | FAM83E |
| 0 | 0.2828 | 0.5267 | 0.0706 | RAB22A |
| 0 | 0.283 | 0.5934 | 0.0936 | EPN2 |
| 0 | 0.2834 | 0.6302 | 0.1097 | KRT5 |
| 0 | 0.2837 | 0.4656 | 0.0873 | FKBP3 |
| 0 | 0.2837 | 0.4049 | 0.1193 | PLD3 |
| 0 | 0.2862 | 0.4718 | 0.1611 | RAF1 |
| 0 | 0.2862 | 0.9851 | 0.1052 | CD24 |
| 0 | 0.2865 | 0.4496 | 0.1157 | CYB561 |
| 0 | 0.2867 | 0.5223 | 0.1209 | RASD2 |
| 0 | 0.2871 | 0.4746 | 0.1387 | MYBL2 |
| 0 | 0.288 | 0.3936 | 0.2119 | URM1 |
| 0 | 0.2889 | 0.5035 | 0.0531 | NDUFB9 |
| 1 | 0.2896 | 0.6973 | 0.0225 | PGM5 |
| 0 | 0.2905 | 0.5741 | 0.059 | MBNL1 |
| 0 | 0.2908 | 0.5972 | 0.1081 | SOX4 |
| 0 | 0.2912 | 0.4792 | 0.1038 | FLJ22659 |
| 0 | 0.2927 | 0.4588 | 0.143 | RRM2 |
| 0 | 0.2934 | 0.548 | 0.0809 | BIRC5 |
| 0 | 0.2936 | 0.6343 | 0.0742 | LSM14A |
| 0 | 0.2938 | 0.5409 | 0.1466 | CDH3 |
| 0 | 0.2942 | 0.5172 | 0.0861 | SUPT6H |
| 0 | 0.2946 | 0.5327 | 0.0864 | SCNN1D |
| 0 | 0.2952 | 0.4817 | 0.1476 | VEGFA |
| 0 | 0.2961 | 0.5157 | 0.1302 | PXN |
| 0 | 0.2982 | 0.9336 | 0.0857 | KLHL25 |
| 0 | 0.2987 | 0.4708 | 0.1476 | MTOR |
| 0 | 0.2995 | 0.5097 | 0.1354 | C9orf58 |
| 0 | 0.2996 | 0.5883 | 0.1308 | GPRIN1 |
| 0 | 0.3012 | 0.5351 | 0.1026 | ANGPTL4 |
| 0 | 0.3025 | 0.6279 | 0.1452 | LSM14A |
| 0 | 0.3031 | 0.5252 | 0.147 | CSNK1D |
| 0 | 0.3045 | 0.6755 | 0.1408 | ADNP |
| 0 | 0.3051 | 0.5116 | 0.192 | ACBD6 |
| 0 | 0.3056 | 0.8967 | 0.1003 | ABCF2 |
| 0 | 0.3064 | 0.4939 | 0.1184 | IDUA |
| 0 | 0.3084 | 0.5233 | 0.1024 | TBX21 |
| 0 | 0.3093 | 0.4011 | 0.1673 | MARVELD2 |
| 0 | 0.3108 | 0.7302 | 0.1545 | RPS25 |
| 0 | 0.3111 | 0.5627 | 0.1679 | SNRP70 |
| 0 | 0.3119 | 0.4873 | 0.1966 | PKP3 |
| 0 | 0.3141 | 0.6169 | 0.1701 | TMEM97 |
| 0 | 0.3144 | 0.5136 | 0.0961 | CFLP1 |
| 0 | 0.3169 | 0.3966 | 0.0784 | BBC3 |
| 0 | 0.3183 | 0.5439 | 0.1563 | TMBIM1 |
| 0 | 0.3186 | 0.5027 | 0.1206 | SLC25A29 |
| 0 | 0.319 | 0.5045 | 0.226 | NXPH3 |
| 0 | 0.3196 | 0.6268 | 0.1121 | MMP11 |
| 0 | 0.3208 | 0.9706 | 0.0888 | ZNF225 |
| 0 | 0.3221 | 0.6674 | 0.1763 | ANLN |
| 0 | 0.323 | 0.5941 | 0.1247 | TMCO1 |
| 0 | 0.3231 | 0.4697 | 0.1157 | ZFP36L1 |
| 0 | 0.3234 | 0.7005 | 0.0918 | LENG8 |
| 0 | 0.3241 | 0.6112 | 0.1165 | ORMDL3 |
| 0 | 0.3294 | 0.4768 | 0.262 | IGH@ |
| 1 | 0.3308 | 0.899 | 0.0292 | UBE2N |
| 0 | 0.3313 | 0.7257 | 0.1573 | IGKV2-24 |
| 0 | 0.3323 | 0.5303 | 0.1746 | EXO1 |
| 0 | 0.3323 | 0.8389 | 0.1382 | PPP2R2D |
| 0 | 0.3338 | 0.6559 | 0.1059 | NDC80 |
| 0 | 0.3348 | 0.5634 | 0.1276 | ELAVL4 |
| 0 | 0.3348 | 0.7748 | 0.1789 | ACBD6 |
| 0 | 0.3352 | 0.5109 | 0.2166 | ENO1 |
| 0 | 0.3352 | 0.5497 | 0.171 | PCBD2 |
| 0 | 0.3362 | 0.8215 | 0.119 | MAFK |
| 0 | 0.3363 | 0.5947 | 0.1581 | ZNF124 |
| 0 | 0.3382 | 0.739 | 0.1145 | SFRS1 |
| 0 | 0.3399 | 0.653 | 0.1391 | GINS2 |
| 0 | 0.3413 | 0.8706 | 0.1732 | DDX42 |
| 0 | 0.3426 | 0.5415 | 0.1592 | RPL34 |
| 0 | 0.3429 | 0.7113 | 0.106 | EHD2 |
| 0 | 0.3436 | 0.5471 | 0.1454 | LOC643159 |
| 0 | 0.3439 | 0.6676 | 0.0879 | C1orf212 |
| 0 | 0.3441 | 0.7145 | 0.091 | MPDU1 |
| 0 | 0.3442 | 0.7819 | 0.1394 | PKP3 |
| 0 | 0.3454 | 0.466 | 0.2367 | FBXW11 |
| 0 | 0.3461 | 0.5657 | 0.1716 | PDZK1IP1 |
| 0 | 0.3465 | 0.4862 | 0.1421 | LOC285830 |
| 0 | 0.3473 | 0.622 | 0.1932 | NDUFB9 |
| 0 | 0.3477 | 0.5942 | 0.0997 | TMEM174 |
| 0 | 0.3491 | 0.7214 | 0.116 | POLR3H |
| 0 | 0.3508 | 0.7058 | 0.0731 | RPS21 |
| 0 | 0.3513 | 0.6841 | 0.2525 | FTH1 |
| 0 | 0.3516 | 0.5412 | 0.1676 | LOC442260 |
| 0 | 0.3517 | 0.9903 | 0.205 | FNDC4 |
| 0 | 0.3524 | 0.6925 | 0.0946 | HN1 |
| 0 | 0.3527 | 0.6956 | 0.2332 | UBTD1 |
| 0 | 0.3532 | 0.6078 | 0.1287 | IGF1R |
| 1 | 0.3536 | 0.8895 | 0.0234 | C16orf42 |
| 0 | 0.3537 | 0.6313 | 0.1657 | ROMO1 |
| 0 | 0.3545 | 0.6306 | 0.1157 | TMEM45B |
| 0 | 0.3554 | 0.5963 | 0.0961 | CLPB |
| 0 | 0.3573 | 0.6657 | 0.1533 | EPOR |
| 0 | 0.3574 | 0.6869 | 0.1213 | HEATR3 |
| 0 | 0.3601 | 0.5999 | 0.1576 | HDGFRP3 |
| 0 | 0.3636 | 0.76 | 0.1202 | TBC1D10B |
| 0 | 0.3649 | 0.6348 | 0.1448 | PGR |
| 0 | 0.3662 | 0.751 | 0.1596 | PYCR1 |
| 0 | 0.3671 | 0.5514 | 0.2303 | C1orf104 |
| 0 | 0.3672 | 0.6094 | 0.2087 | FAM148A |
| 0 | 0.3672 | 0.6682 | 0.1416 | TAF2 |
| 0 | 0.3678 | 0.8589 | 0.1345 | CCL21 |
| 0 | 0.3682 | 0.611 | 0.1873 | LARS |
| 0 | 0.3704 | 0.5236 | 0.1487 | PRELID1 |
| 0 | 0.3708 | 0.7718 | 0.1132 | PLEKHF2 |
| 0 | 0.3721 | 0.9015 | 0.1233 | MDM2 |
| 0 | 0.3737 | 0.6432 | 0.0805 | HN1 |
| 0 | 0.3752 | 0.7665 | 0.1833 | GATA3 |
| 0 | 0.3753 | 0.5781 | 0.1844 | RND3 |
| 0 | 0.3754 | 0.4618 | 0.2416 | RAF1 |
| 0 | 0.3758 | 0.9211 | 0.1171 | DRD2 |
| 0 | 0.3766 | 0.6449 | 0.1948 | LOC442270 |
| 0 | 0.3773 | 0.5883 | 0.1959 | ADCY7 |
| 0 | 0.3795 | 0.7988 | 0.1846 | BAG1 |
| 0 | 0.3805 | 0.6565 | 0.1112 | FBXO15 |
| 0 | 0.3805 | 0.711 | 0.2165 | MLPH |
| 0 | 0.3806 | 0.7101 | 0.1911 | FLAD1 |
| 0 | 0.3807 | 0.7426 | 0.192 | LOC730275 |
| 0 | 0.3813 | 0.614 | 0.1423 | KIAA0310 |
| 0 | 0.3814 | 0.6025 | 0.1592 | LASS6 |
| 0 | 0.3816 | 0.6542 | 0.1053 | LOC442260 |
| 0 | 0.3817 | 0.57 | 0.1464 | IGJ |
| 0 | 0.3823 | 0.7164 | 0.1995 | C15orf52 |
| 0 | 0.3828 | 0.8011 | 0.2048 | CAPS |
| 0 | 0.3853 | 0.7792 | 0.1397 | GPR160 |
| 0 | 0.3855 | 0.6771 | 0.2797 | RPLP0 |
| 0 | 0.3861 | 0.6249 | 0.1425 | B4GALT1 |
| 0 | 0.3861 | 0.6816 | 0.1504 | SLC39A6 |
| 0 | 0.3865 | 0.9027 | 0.0602 | MKI67 |
| 0 | 0.3868 | 0.6311 | 0.2163 | SNRP70 |
| 0 | 0.3895 | 0.6042 | 0.2299 | UBE2T |
| 0 | 0.3896 | 0.8027 | 0.2385 | CCDC25 |
| 0 | 0.3905 | 0.5502 | 0.0701 | KIF2C |
| 0 | 0.3924 | 0.7239 | 0.1798 | PYCR1 |
| 0 | 0.3936 | 0.6936 | 0.1433 | CSNK1D |
| 0 | 0.3945 | 0.5928 | 0.2017 | HYPK |
| 0 | 0.3964 | 0.8984 | 0.1292 | ISOC1 |
| 0 | 0.3979 | 0.8908 | 0.0577 | HNRPAB |
| 0 | 0.3984 | 0.5033 | 0.317 | MBOAT2 |
| 0 | 0.3991 | 0.8319 | 0.121 | PGM5 |
| 0 | 0.4004 | 0.555 | 0.1985 | MMP11 |
| 0 | 0.4004 | 0.6644 | 0.1864 | TMSB10 |
| 0 | 0.4007 | 0.7334 | 0.1492 | ADNP |
| 0 | 0.4011 | 0.9234 | 0.1192 | GAMT |
| 0 | 0.4015 | 0.575 | 0.1961 | MBOAT2 |
| 0 | 0.4016 | 0.8532 | 0.1565 | ETS2 |
| 0 | 0.4016 | 0.9722 | 0.1626 | PRPF40A |
| 0 | 0.4024 | 0.619 | 0.2141 | PADI2 |
| 0 | 0.4029 | 0.5256 | 0.2863 | MIA |
| 0 | 0.4046 | 0.7492 | 0.1653 | MRPS12 |
| 0 | 0.405 | 0.789 | 0.2283 | LOC400590 |
| 0 | 0.4062 | 0.8739 | 0.2331 | GALNT2 |
| 0 | 0.407 | 0.6108 | 0.1169 | MTOR |
| 0 | 0.4086 | 0.751 | 0.2249 | CYBRD1 |
| 0 | 0.4088 | 0.8071 | 0.1578 | RASD2 |
| 0 | 0.411 | 0.7435 | 0.1713 | LOC401397 |
| 0 | 0.4112 | 0.8497 | 0.1897 | SLC6A19 |
| 0 | 0.4123 | 0.6129 | 0.2875 | UBTD1 |
| 0 | 0.4123 | 0.7862 | 0.171 | CLIP3 |
| 0 | 0.413 | 0.6012 | 0.1462 | MGST3 |
| 0 | 0.4138 | 0.7788 | 0.1071 | AGPS |
| 0 | 0.4138 | 0.9223 | 0.1197 | CDC20 |
| 0 | 0.4166 | 0.7074 | 0.2572 | GSTM1 |
| 0 | 0.4177 | 0.693 | 0.2366 | THRAP1 |
| 0 | 0.4188 | 0.7162 | 0.1654 | GINS2 |
| 0 | 0.4188 | 0.7825 | 0.0888 | RPL23A |
| 0 | 0.42 | 0.6814 | 0.2875 | MGST3 |
| 0 | 0.4201 | 0.6393 | 0.191 | VPRBP |
| 0 | 0.4206 | 0.8924 | 0.0643 | ARL8A |
| 0 | 0.421 | 0.5552 | 0.2112 | RPS25 |
| 0 | 0.4218 | 0.8356 | 0.1252 | KRTAP5-9 |
| 0 | 0.4218 | 0.8082 | 0.2512 | C16orf42 |
| 0 | 0.4234 | 0.7464 | 0.1311 | PSMC5 |
| 0 | 0.4241 | 0.9274 | 0.1459 | CTSL2 |
| 0 | 0.4243 | 0.7231 | 0.2217 | DHPS |
| 0 | 0.4256 | 0.8829 | 0.2063 | ADORA3 |
| 0 | 0.4261 | 0.83 | 0.2359 | MFSD1 |
| 0 | 0.4273 | 0.7555 | 0.1193 | VPS18 |
| 0 | 0.4284 | 0.7952 | 0.1388 | SLC25A31 |
| 0 | 0.4284 | 0.8508 | 0.1151 | UBFD1 |
| 0 | 0.4286 | 0.8975 | 0.1875 | C1QL2 |
| 0 | 0.4287 | 0.5217 | 0.3514 | IDUA |
| 0 | 0.4291 | 0.7443 | 0.1741 | STK11IP |
| 0 | 0.4295 | 0.8537 | 0.1405 | ARL17 |
| 0 | 0.4299 | 0.7508 | 0.1631 | FLAD1 |
| 0 | 0.433 | 0.9939 | 0.2011 | NDC80 |
| 0 | 0.434 | 0.7919 | 0.1512 | KRTAP2-4 |
| 0 | 0.4342 | 0.671 | 0.1749 | KRT18 |
| 0 | 0.4343 | 0.7493 | 0.118 | PGRMC2 |
| 0 | 0.4354 | 0.9375 | 0.1572 | MSI2 |
| 0 | 0.4359 | 0.6397 | 0.2313 | TRABD |
| 0 | 0.4362 | 0.7742 | 0.1324 | ROMO1 |
| 0 | 0.4366 | 0.7986 | 0.1156 | PCBD2 |
| 0 | 0.4367 | 0.9087 | 0.2691 | BCAS1 |
| 0 | 0.4368 | 0.7495 | 0.186 | BRD2 |
| 0 | 0.4369 | 0.6463 | 0.1931 | IGHA1 |
| 0 | 0.4371 | 0.8285 | 0.1683 | MAZ |
| 0 | 0.4385 | 0.7279 | 0.2212 | FGFR4 |
| 0 | 0.4386 | 0.5905 | 0.202 | CD24 |
| 0 | 0.4388 | 0.7507 | 0.1749 | AK1 |
| 0 | 0.439 | 0.7462 | 0.1921 | TBX10 |
| 0 | 0.4395 | 0.8645 | 0.1521 | GAMT |
| 0 | 0.4401 | 0.8157 | 0.2912 | LOC442260 |
| 0 | 0.4402 | 0.7576 | 0.1101 | UBE2N |
| 0 | 0.4411 | 0.9307 | 0.1845 | FNDC4 |
| 0 | 0.4411 | 0.688 | 0.2201 | SUPT6H |
| 0 | 0.4412 | 0.8623 | 0.091 | CCDC74A |
| 0 | 0.4438 | 0.8942 | 0.1259 | SFRS1 |
| 0 | 0.4462 | 0.7729 | 0.1764 | RPS14 |
| 0 | 0.4462 | 0.8168 | 0.125 | C16orf14 |
| 0 | 0.4463 | 0.9565 | 0.0946 | KRTAP2-4 |
| 0 | 0.4466 | 0.6565 | 0.1693 | BBC3 |
| 0 | 0.4469 | 0.803 | 0.1084 | FAM84B |
| 0 | 0.447 | 0.845 | 0.2457 | ZACN |
| 0 | 0.4507 | 0.7808 | 0.1479 | ZNF704 |
| 0 | 0.4508 | 0.9147 | 0.2458 | VDAC1 |
| 0 | 0.4515 | 0.7983 | 0.1594 | MRPS36 |
| 0 | 0.452 | 0.7063 | 0.2498 | SSBP2 |
| 0 | 0.4527 | 0.6211 | 0.1544 | TBC1D9 |
| 0 | 0.4531 | 0.9055 | 0.2287 | RPAP1 |
| 0 | 0.4539 | 0.8174 | 0.1172 | C15orf52 |
| 0 | 0.4548 | 0.9576 | 0.1074 | ORC6L |
| 0 | 0.4569 | 0.8157 | 0.1044 | ADNP |
| 0 | 0.4595 | 0.7648 | 0.1369 | EPOR |
| 0 | 0.4611 | 0.9802 | 0.1373 | KGFLP1 |
| 0 | 0.4612 | 0.9553 | 0.1917 | ARL8A |
| 0 | 0.4612 | 0.7023 | 0.2153 | DDX42 |
| 0 | 0.4613 | 0.6274 | 0.3089 | KRTAP19-1 |
| 0 | 0.4618 | 0.9914 | 0.1633 | FLOT2 |
| 0 | 0.4619 | 0.6638 | 0.2464 | NEDD8 |
| 0 | 0.4641 | 0.7182 | 0.2446 | C20orf67 |
| 0 | 0.4647 | 0.8964 | 0.1143 | LOC642852 |
| 0 | 0.4663 | 0.8471 | 0.2224 | HYPK |
| 0 | 0.4672 | 0.7489 | 0.1967 | LAYN |
| 0 | 0.469 | 0.8757 | 0.2289 | CCDC25 |
| 0 | 0.4693 | 0.6757 | 0.1736 | OGFR |
| 0 | 0.4712 | 0.7487 | 0.2692 | RPS14 |
| 0 | 0.4715 | 0.9546 | 0.1425 | MTOR |
| 0 | 0.472 | 0.8737 | 0.1708 | FLOT2 |
| 0 | 0.4738 | 0.9688 | 0.1734 | PXN |
| 0 | 0.4742 | 0.9384 | 0.0861 | SLC25A5 |
| 0 | 0.4747 | 0.7689 | 0.2258 | PLD3 |
| 0 | 0.4753 | 0.7034 | 0.2917 | TMEM45B |
| 0 | 0.4754 | 0.6429 | 0.2767 | CUGBP1 |
| 0 | 0.4763 | 0.7979 | 0.2544 | MRPS36 |
| 0 | 0.4763 | 0.9343 | 0.1583 | ZNF704 |
| 0 | 0.4766 | 0.9207 | 0.2153 | CCDC24 |
| 0 | 0.4767 | 0.864 | 0.2005 | SMG1 |
| 0 | 0.477 | 0.9732 | 0.1225 | UBFD1 |
| 0 | 0.4783 | 0.6856 | 0.1954 | ADORA3 |
| 0 | 0.4799 | 0.6935 | 0.2601 | SLC25A28 |
| 0 | 0.4812 | 0.9412 | 0.1861 | NME3 |
| 0 | 0.4813 | 0.839 | 0.1565 | HNRNPA1L2 |
| 0 | 0.4825 | 0.6575 | 0.1904 | KCNE4 |
| 0 | 0.4843 | 0.9812 | 0.1786 | LCE3E |
| 0 | 0.4846 | 0.7526 | 0.2009 | SLC25A29 |
| 0 | 0.4847 | 0.7087 | 0.3026 | LOC649178 |
| 0 | 0.4851 | 0.6757 | 0.3091 | GPR160 |
| 0 | 0.4855 | 0.7326 | 0.1245 | PRR3 |
| 0 | 0.4858 | 0.9316 | 0.3019 | LOC285830 |
| 0 | 0.4861 | 0.7334 | 0.1708 | IGKV1-5 |
| 0 | 0.4862 | 0.7291 | 0.2521 | C19orf28 |
| 0 | 0.4874 | 0.8667 | 0.284 | CSNK1A1 |
| 0 | 0.4877 | 0.7902 | 0.2337 | UBE2W |
| 0 | 0.4883 | 0.8346 | 0.2097 | POGZ |
| 0 | 0.4888 | 0.8615 | 0.1317 | DPY19L4 |
| 0 | 0.4901 | 0.9288 | 0.2347 | CXXC5 |
| 0 | 0.4915 | 0.8431 | 0.198 | MGC24125 |
| 0 | 0.4933 | 0.8563 | 0.3301 | KGFLP1 |
| 0 | 0.4934 | 0.7928 | 0.225 | TP53 |
| 0 | 0.4936 | 0.8377 | 0.1802 | PTK2 |
| 0 | 0.4944 | 0.8622 | 0.2002 | GPR22 |
| 0 | 0.4952 | 0.9651 | 0.1877 | EXO1 |
| 0 | 0.496 | 0.9096 | 0.2492 | KRT17 |
| 0 | 0.4965 | 0.8691 | 0.2708 | DDX42 |
| 0 | 0.4971 | 0.956 | 0.2376 | RPS28 |
| 0 | 0.4973 | 0.9344 | 0.2066 | ERBB4 |
| 0 | 0.4978 | 0.9956 | 0.2911 | CLIP3 |
| 0 | 0.4987 | 0.7218 | 0.313 | PTK2 |
| 0 | 0.5001 | 0.9207 | 0.2011 | GSN |
| 0 | 0.5004 | 0.9599 | 0.2139 | KIAA1815 |
| 0 | 0.5013 | 0.8068 | 0.3743 | STEAP3 |
| 0 | 0.5017 | 0.8404 | 0.2708 | KRT18P28 |
| 0 | 0.5039 | 0.7031 | 0.2445 | RASSF7 |
| 0 | 0.5042 | 0.9162 | 0.1926 | PLD4 |
| 0 | 0.5046 | 0.9301 | 0.2515 | ADCYAP1 |
| 0 | 0.5054 | 0.9395 | 0.2081 | LSMD1 |
| 0 | 0.5063 | 0.7963 | 0.204 | NXPH3 |
| 0 | 0.5064 | 0.8855 | 0.1803 | SLC6A19 |
| 0 | 0.5066 | 0.717 | 0.3463 | STEAP3 |
| 0 | 0.5086 | 0.9977 | 0.1348 | TBC1D10B |
| 0 | 0.5093 | 0.9829 | 0.1077 | CDC6 |
| 0 | 0.51 | 0.8542 | 0.1738 | TAPBP |
| 0 | 0.5101 | 0.7863 | 0.2278 | HNRNPA1L2 |
| 0 | 0.5109 | 0.9359 | 0.1779 | CCNB1 |
| 0 | 0.511 | 0.9949 | 0.2187 | AURKA |
| 0 | 0.5118 | 0.6619 | 0.2239 | VPS37B |
| 0 | 0.5119 | 0.977 | 0.1873 | DPY19L4 |
| 0 | 0.5128 | 0.9219 | 0.0507 | MGC4093 |
| 0 | 0.5132 | 0.8587 | 0.3225 | SLC30A10 |
| 0 | 0.5137 | 0.6977 | 0.3608 | EHMT1 |
| 0 | 0.5158 | 0.8182 | 0.3438 | TFRC |
| 0 | 0.5159 | 0.8333 | 0.2781 | CCDC24 |
| 0 | 0.5164 | 0.9912 | 0.1939 | RCL1 |
| 0 | 0.5171 | 0.8637 | 0.1361 | KRT14 |
| 0 | 0.5187 | 0.9362 | 0.1708 | MTCH2 |
| 0 | 0.52 | 0.8154 | 0.2767 | SPTAN1 |
| 0 | 0.521 | 0.9374 | 0.1112 | BDH2 |
| 0 | 0.5211 | 0.7856 | 0.2716 | VPS37B |
| 0 | 0.5222 | 0.8935 | 0.2842 | KRT18 |
| 0 | 0.5224 | 0.9437 | 0.2132 | DRD2 |
| 0 | 0.5233 | 0.9794 | 0.2427 | SLC6A19 |
| 0 | 0.524 | 0.7061 | 0.374 | ADFP |
| 0 | 0.5259 | 0.8753 | 0.1395 | LOC346887 |
| 0 | 0.5264 | 0.8196 | 0.2214 | HIBCH |
| 0 | 0.5264 | 0.759 | 0.2759 | TCEB2 |
| 0 | 0.5267 | 0.8973 | 0.3408 | MBNL1 |
| 0 | 0.5274 | 0.9924 | 0.2231 | HNRNPA1L2 |
| 0 | 0.5275 | 0.6931 | 0.4012 | PHGDH |
| 0 | 0.5285 | 0.8256 | 0.2666 | KHSRP |
| 0 | 0.5298 | 0.8335 | 0.4093 | ELN |
| 0 | 0.5317 | 0.9032 | 0.3174 | CUGBP1 |
| 0 | 0.532 | 0.7359 | 0.2556 | PHB2 |
| 0 | 0.5322 | 0.9112 | 0.1902 | UGDH |
| 0 | 0.5325 | 0.8362 | 0.1965 | MPDU1 |
| 0 | 0.5327 | 0.8594 | 0.2145 | CCNB1 |
| 0 | 0.5329 | 0.9997 | 0.2088 | TMEM121 |
| 0 | 0.5337 | 0.7695 | 0.2558 | SCUBE2 |
| 0 | 0.5338 | 0.7466 | 0.3408 | CCDC25 |
| 0 | 0.5339 | 0.934 | 0.0906 | MGC4093 |
| 0 | 0.5345 | 0.7322 | 0.2295 | TBC1D9 |
| 0 | 0.5347 | 0.9482 | 0.2669 | FABP5 |
| 0 | 0.5352 | 0.833 | 0.2314 | POM121L9P |
| 0 | 0.536 | 0.8139 | 0.286 | SPDEF |
| 0 | 0.5364 | 0.6418 | 0.3183 | SNX11 |
| 0 | 0.5365 | 0.6782 | 0.3338 | ARHGEF11 |
| 0 | 0.5366 | 0.9142 | 0.2159 | IFI27L1 |
| 0 | 0.5366 | 0.8427 | 0.2727 | KRTAP13-2 |
| 0 | 0.5377 | 0.9448 | 0.2555 | BLVRA |
| 0 | 0.5379 | 0.8465 | 0.2358 | MAP3K13 |
| 0 | 0.5379 | 0.9508 | 0.1348 | DKFZP434A0131 |
| 0 | 0.5392 | 0.9921 | 0.0838 | RCL1 |
| 0 | 0.5393 | 0.8098 | 0.3437 | MSI2 |
| 0 | 0.5397 | 0.8193 | 0.2463 | NECAB3 |
| 0 | 0.5404 | 0.904 | 0.1486 | SLC16A8 |
| 0 | 0.5405 | 0.7599 | 0.1872 | FARP2 |
| 0 | 0.5407 | 0.9228 | 0.2023 | RPS3A |
| 0 | 0.5416 | 0.8974 | 0.2815 | PGRMC2 |
| 0 | 0.5432 | 0.7967 | 0.2505 | KCNE4 |
| 0 | 0.5436 | 0.9841 | 0.1934 | IGHV1-69 |
| 0 | 0.5437 | 0.9798 | 0.0918 | SFRP1 |
| 0 | 0.5443 | 0.9506 | 0.2256 | SLC30A10 |
| 0 | 0.5446 | 0.9415 | 0.1843 | FOXC1 |
| 0 | 0.545 | 0.9833 | 0.2468 | CXorf56 |
| 0 | 0.5465 | 0.9348 | 0.3156 | SMS |
| 0 | 0.548 | 0.873 | 0.259 | MIA |
| 0 | 0.5503 | 0.7081 | 0.3715 | Kua-UEV |
| 0 | 0.5509 | 0.9427 | 0.2898 | UGDH |
| 0 | 0.552 | 0.9927 | 0.2002 | RPS3A |
| 0 | 0.5524 | 0.9194 | 0.1359 | UBE2C |
| 0 | 0.5525 | 0.8963 | 0.2301 | FAM110A |
| 0 | 0.5527 | 0.8168 | 0.3731 | LARS |
| 0 | 0.5531 | 0.7951 | 0.3719 | RND3 |
| 0 | 0.5532 | 0.9411 | 0.318 | SPP1 |
| 0 | 0.5542 | 0.8005 | 0.297 | C19orf28 |
| 0 | 0.5552 | 0.9352 | 0.2419 | FAM83E |
| 0 | 0.5555 | 0.9513 | 0.3464 | PDZK1IP1 |
| 0 | 0.5555 | 0.9528 | 0.3324 | PHRF1 |
| 0 | 0.556 | 0.9262 | 0.3201 | PRPF40A |
| 0 | 0.558 | 0.8458 | 0.25 | RAB22A |
| 0 | 0.5586 | 0.753 | 0.2644 | FABP5 |
| 0 | 0.5592 | 0.9235 | 0.275 | RELB |
| 0 | 0.5599 | 0.9114 | 0.3087 | HSPA8 |
| 0 | 0.56 | 0.8345 | 0.2174 | ATAD3A |
| 0 | 0.5604 | 0.9916 | 0.2094 | YWHAZ |
| 0 | 0.5604 | 0.9009 | 0.1947 | CLPB |
| 0 | 0.5623 | 0.9171 | 0.3045 | TEX2 |
| 0 | 0.5632 | 0.961 | 0.3307 | DNAJC4 |
| 0 | 0.5635 | 0.95 | 0.2629 | SPTAN1 |
| 0 | 0.564 | 0.9526 | 0.1988 | SULT1A2 |
| 0 | 0.5641 | 0.9053 | 0.1616 | FNDC4 |
| 0 | 0.565 | 0.9711 | 0.1287 | BCAS1 |
| 0 | 0.5667 | 0.7795 | 0.2515 | PGR |
| 0 | 0.5673 | 0.9139 | 0.2956 | MALAT1 |
| 0 | 0.5688 | 0.8143 | 0.3014 | LOC200810 |
| 0 | 0.5698 | 0.9129 | 0.3094 | KCNE4 |
| 0 | 0.5699 | 0.8543 | 0.2923 | TMEM97 |
| 0 | 0.5703 | 0.9738 | 0.2347 | IGKV1-5 |
| 0 | 0.5704 | 0.9449 | 0.3253 | UNC119 |
| 0 | 0.5714 | 0.938 | 0.2877 | DAD1 |
| 0 | 0.5724 | 0.8395 | 0.3078 | ZC3HAV1L |
| 0 | 0.5727 | 0.9818 | 0.2937 | GPR22 |
| 0 | 0.573 | 0.8676 | 0.311 | MAZ |
| 0 | 0.573 | 0.8893 | 0.2804 | PYCR1 |
| 0 | 0.5746 | 0.9841 | 0.2752 | TP53 |
| 0 | 0.5755 | 0.7404 | 0.0867 | MAD2L2 |
| 0 | 0.5759 | 0.8088 | 0.214 | METTL3 |
| 0 | 0.577 | 0.9375 | 0.294 | FRMD4A |
| 0 | 0.5773 | 0.961 | 0.0755 | GALNT10 |
| 0 | 0.5775 | 0.888 | 0.3911 | HPS6 |
| 0 | 0.5781 | 0.8532 | 0.2051 | MFSD1 |
| 0 | 0.5801 | 0.9834 | 0.2924 | KIAA0146 |
| 0 | 0.5801 | 0.9337 | 0.3372 | PCK2 |
| 0 | 0.5804 | 0.8573 | 0.4032 | GPRIN1 |
| 0 | 0.5806 | 0.8192 | 0.2239 | IFI27L1 |
| 0 | 0.5809 | 0.9849 | 0.2686 | MAFK |
| 0 | 0.5826 | 0.9883 | 0.0957 | RPL10 |
| 0 | 0.5832 | 0.8703 | 0.2143 | NECAB3 |
| 0 | 0.584 | 0.8789 | 0.2226 | BTG2 |
| 0 | 0.5841 | 0.9721 | 0.327 | KIAA0146 |
| 0 | 0.5842 | 0.9272 | 0.2407 | LOC652261 |
| 0 | 0.5853 | 0.9545 | 0.2107 | PHRF1 |
| 0 | 0.5854 | 0.9175 | 0.2564 | ST6GALNAC4 |
| 0 | 0.5856 | 0.9715 | 0.1879 | RPS21 |
| 0 | 0.5872 | 0.849 | 0.3134 | UBR2 |
| 0 | 0.5881 | 0.9613 | 0.2782 | KIAA0652 |
| 0 | 0.5881 | 0.8298 | 0.3702 | MVP |
| 0 | 0.5885 | 0.8047 | 0.2628 | TCEB2 |
| 0 | 0.5885 | 0.8458 | 0.34 | CHD6 |
| 0 | 0.5886 | 0.9733 | 0.3825 | TMEM19 |
| 0 | 0.5902 | 0.9988 | 0.2531 | FRAG1 |
| 0 | 0.5903 | 0.7583 | 0.2754 | CAPS |
| 0 | 0.5917 | 0.9714 | 0.3515 | CLPP |
| 0 | 0.5918 | 0.9612 | 0.3871 | HIST2H2BE |
| 0 | 0.5937 | 0.8122 | 0.1991 | KIAA1920 |
| 0 | 0.594 | 0.8599 | 0.3944 | NXPH3 |
| 0 | 0.596 | 0.9099 | 0.2733 | PKP3 |
| 0 | 0.596 | 0.9531 | 0.3264 | C14orf1 |
| 0 | 0.5961 | 0.8943 | 0.3773 | FBXO25 |
| 0 | 0.5962 | 0.9994 | 0.3779 | GINS2 |
| 0 | 0.5963 | 0.9871 | 0.2586 | CYB561 |
| 0 | 0.5964 | 0.7641 | 0.3282 | KIAA1920 |
| 0 | 0.5971 | 0.917 | 0.2213 | YWHAZ |
| 0 | 0.5974 | 0.9979 | 0.2618 | VEGFA |
| 0 | 0.5976 | 0.9969 | 0.2048 | THOP1 |
| 0 | 0.5985 | 0.8242 | 0.2282 | MMP11 |
| 0 | 0.5987 | 0.885 | 0.2829 | EMP2 |
| 0 | 0.599 | 0.981 | 0.2009 | ERBB2IP |
| 0 | 0.5991 | 0.9446 | 0.2839 | CHD6 |
| 0 | 0.6009 | 0.923 | 0.2616 | SCNN1D |
| 0 | 0.6021 | 0.9153 | 0.1722 | MAD2L2 |
| 0 | 0.6025 | 0.9562 | 0.4342 | CNFN |
| 0 | 0.6026 | 0.8597 | 0.3629 | EPN2 |
| 0 | 0.6027 | 0.9924 | 0.2293 | METTL3 |
| 0 | 0.6035 | 0.9529 | 0.3295 | ENPP1 |
| 0 | 0.6038 | 0.9669 | 0.2092 | CFLP1 |
| 0 | 0.6054 | 0.9875 | 0.3096 | SLAIN2 |
| 0 | 0.6056 | 0.8544 | 0.329 | DDX50 |
| 0 | 0.6083 | 0.9704 | 0.1802 | CDC6 |
| 0 | 0.6091 | 0.9572 | 0.355 | RBM14 |
| 0 | 0.6092 | 0.9814 | 0.2331 | POGZ |
| 0 | 0.6097 | 0.9903 | 0.2645 | MMD |
| 0 | 0.6097 | 0.9724 | 0.3251 | RPS3A |
| 0 | 0.6097 | 0.9948 | 0.2198 | CXXC5 |
| 0 | 0.6119 | 0.8747 | 0.3061 | ZNF609 |
| 0 | 0.6121 | 0.9526 | 0.2149 | CLNS1A |
| 0 | 0.6124 | 0.9051 | 0.2536 | AURKA |
| 0 | 0.6125 | 0.9951 | 0.2624 | MAPT |
| 0 | 0.6125 | 0.9932 | 0.1133 | BDH2 |
| 0 | 0.6125 | 0.923 | 0.2215 | ZC3H15 |
| 0 | 0.6126 | 0.9195 | 0.2967 | LOC442019 |
| 0 | 0.6141 | 0.9381 | 0.3814 | EGFR |
| 0 | 0.6143 | 0.9516 | 0.1436 | GHR |
| 0 | 0.6146 | 0.9331 | 0.4167 | BMP2K |
| 0 | 0.6148 | 0.8948 | 0.1792 | SMARCD2 |
| 0 | 0.6159 | 0.9354 | 0.2584 | ACTR3B |
| 0 | 0.6161 | 0.9631 | 0.2091 | IMPAD1 |
| 0 | 0.6162 | 0.9736 | 0.3626 | LOC401397 |
| 0 | 0.6165 | 0.9373 | 0.4053 | RBM14 |
| 0 | 0.6166 | 0.9981 | 0.3839 | TUBB2C |
| 0 | 0.6169 | 0.894 | 0.3824 | PTEN |
| 0 | 0.617 | 0.8699 | 0.412 | AKT1 |
| 0 | 0.6171 | 0.921 | 0.3615 | LOC285830 |
| 0 | 0.6173 | 0.8837 | 0.3683 | RHBDD1 |
| 0 | 0.6174 | 0.9658 | 0.2774 | TEX2 |
| 0 | 0.6176 | 0.9403 | 0.296 | RPS14 |
| 0 | 0.6186 | 0.913 | 0.3621 | MPDU1 |
| 0 | 0.6187 | 0.8494 | 0.3357 | TP53 |
| 0 | 0.6188 | 0.8623 | 0.1966 | LOC653391 |
| 0 | 0.6194 | 0.997 | 0.2664 | RAB27B |
| 0 | 0.6196 | 0.8972 | 0.1835 | FAM84B |
| 0 | 0.6205 | 0.7705 | 0.379 | PSMD3 |
| 0 | 0.6205 | 0.946 | 0.3166 | RPS28 |
| 0 | 0.621 | 0.9781 | 0.2499 | KGFLP1 |
| 0 | 0.6212 | 0.9183 | 0.2461 | ZC3HAV1L |
| 0 | 0.6212 | 0.8697 | 0.3782 | CLIC1 |
| 0 | 0.6218 | 0.8033 | 0.3738 | CRTC2 |
| 0 | 0.6224 | 0.9059 | 0.2255 | MALAT1 |
| 0 | 0.6233 | 0.9923 | 0.3238 | MDM2 |
| 0 | 0.6234 | 0.7724 | 0.3386 | PTTG1 |
| 0 | 0.6237 | 0.8941 | 0.2905 | POM121L9P |
| 0 | 0.6253 | 0.89 | 0.1562 | ABCF2 |
| 0 | 0.6255 | 0.8224 | 0.3288 | ELN |
| 0 | 0.6259 | 0.9521 | 0.4245 | LENG8 |
| 0 | 0.6266 | 0.9074 | 0.4328 | LOC649178 |
| 0 | 0.6267 | 0.9657 | 0.3544 | FKBP3 |
| 0 | 0.627 | 0.9617 | 0.4084 | TCEB2 |
| 0 | 0.6272 | 0.9282 | 0.2686 | SLC39A6 |
| 0 | 0.6276 | 0.9633 | 0.4689 | ST6GALNAC4 |
| 0 | 0.628 | 0.9829 | 0.3149 | CLPP |
| 0 | 0.628 | 0.8813 | 0.2685 | DDX50 |
| 0 | 0.6281 | 0.9477 | 0.3428 | XYLT1 |
| 0 | 0.6289 | 0.9378 | 0.3864 | GALNT2 |
| 0 | 0.6313 | 0.9804 | 0.2038 | KRT14 |
| 0 | 0.6314 | 0.9661 | 0.2993 | SOX4 |
| 0 | 0.633 | 0.9948 | 0.3844 | AKT1 |
| 0 | 0.6331 | 0.9148 | 0.4279 | C17orf37 |
| 0 | 0.634 | 0.986 | 0.2757 | PRELID1 |
| 0 | 0.6347 | 0.862 | 0.5175 | VPS37B |
| 0 | 0.6349 | 0.9252 | 0.3608 | CENPF |
| 0 | 0.6356 | 0.7589 | 0.4233 | SNRP70 |
| 0 | 0.6364 | 0.8009 | 0.4371 | SCNN1D |
| 0 | 0.6367 | 0.8482 | 0.3367 | HDGFRP3 |
| 0 | 0.6367 | 0.9998 | 0.3151 | THRAP1 |
| 0 | 0.637 | 0.9236 | 0.3972 | PRKD3 |
| 0 | 0.6376 | 0.9146 | 0.4745 | SREBF2 |
| 0 | 0.6376 | 0.9847 | 0.2059 | C1QL2 |
| 0 | 0.6377 | 0.8456 | 0.3928 | ERBB2 |
| 0 | 0.6379 | 0.9837 | 0.2563 | MSN |
| 0 | 0.6381 | 0.9377 | 0.3416 | SELO |
| 0 | 0.6382 | 0.8633 | 0.3039 | CACNG7 |
| 0 | 0.6388 | 0.9836 | 0.1054 | VDAC1 |
| 0 | 0.6402 | 0.9715 | 0.2158 | TEX2 |
| 0 | 0.6404 | 0.8321 | 0.4685 | FBXO25 |
| 0 | 0.6409 | 0.9168 | 0.223 | RPL10 |
| 0 | 0.6409 | 0.8981 | 0.284 | DAD1 |
| 0 | 0.6412 | 0.9677 | 0.4101 | CAPS |
| 0 | 0.642 | 0.957 | 0.2921 | H2AFY |
| 0 | 0.642 | 0.9992 | 0.331 | ILF2 |
| 0 | 0.642 | 0.9431 | 0.0661 | RPS21 |
| 0 | 0.643 | 0.9307 | 0.4789 | TFRC |
| 0 | 0.6433 | 0.9527 | 0.3386 | PTK2 |
| 0 | 0.6449 | 0.956 | 0.362 | BRD2 |
| 0 | 0.6458 | 0.9681 | 0.3661 | RPS2 |
| 0 | 0.6464 | 0.8606 | 0.265 | KRTAP2-4 |
| 0 | 0.6466 | 0.9062 | 0.4614 | UBFD1 |
| 0 | 0.6468 | 0.9405 | 0.4443 | IRGC |
| 0 | 0.6474 | 0.9228 | 0.479 | IGHA1 |
| 0 | 0.648 | 0.8942 | 0.3385 | SPDEF |
| 0 | 0.65 | 0.8887 | 0.255 | MYBL2 |
| 0 | 0.6508 | 0.9669 | 0.3298 | FGFR4 |
| 0 | 0.651 | 0.9915 | 0.1339 | PGM5 |
| 0 | 0.652 | 0.9904 | 0.4144 | HIST2H2BE |
| 0 | 0.6523 | 0.914 | 0.3021 | SF3B3 |
| 0 | 0.6523 | 0.917 | 0.3656 | DHPS |
| 0 | 0.6528 | 0.9862 | 0.351 | FRMD4A |
| 0 | 0.6532 | 0.8503 | 0.3567 | TBC1D10B |
| 0 | 0.6533 | 0.9472 | 0.3838 | GSN |
| 0 | 0.6535 | 0.9044 | 0.3562 | NUF2 |
| 0 | 0.6538 | 0.8938 | 0.3959 | GAMT |
| 0 | 0.6539 | 0.9385 | 0.3718 | PCK2 |
| 0 | 0.6544 | 0.8728 | 0.4005 | FRMD4A |
| 0 | 0.6546 | 0.9983 | 0.1605 | IGJ |
| 0 | 0.6552 | 0.999 | 0.4203 | RRM2 |
| 0 | 0.6554 | 0.8896 | 0.4308 | ANGPTL2 |
| 0 | 0.6554 | 0.9756 | 0.3819 | BDH2 |
| 0 | 0.6555 | 0.9101 | 0.2109 | AGPS |
| 0 | 0.6555 | 0.9767 | 0.2594 | CIAPIN1 |
| 0 | 0.6556 | 0.8917 | 0.431 | ALG13 |
| 0 | 0.6559 | 0.9564 | 0.3499 | PRKD3 |
| 0 | 0.6561 | 0.842 | 0.3549 | GUSB |
| 0 | 0.6565 | 0.8903 | 0.4051 | SLC16A8 |
| 0 | 0.6565 | 0.9731 | 0.4523 | ACTR3B |
| 0 | 0.657 | 0.8964 | 0.3038 | PRR3 |
| 0 | 0.6579 | 0.9279 | 0.4306 | PTPRA |
| 0 | 0.6595 | 0.8248 | 0.3348 | NME4 |
| 0 | 0.6601 | 0.8765 | 0.4759 | ACTR3B |
| 0 | 0.6617 | 0.9884 | 0.2203 | ACAD9 |
| 0 | 0.6618 | 0.8665 | 0.4001 | FLJ22659 |
| 0 | 0.6624 | 0.9275 | 0.3648 | BCL2 |
| 0 | 0.664 | 0.999 | 0.3043 | ARL17 |
| 0 | 0.6651 | 0.9212 | 0.4236 | SNHG5 |
| 0 | 0.666 | 0.9938 | 0.2815 | MMD |
| 0 | 0.6662 | 0.9768 | 0.2878 | JMJD1B |
| 0 | 0.6663 | 0.9424 | 0.385 | HYPK |
| 0 | 0.6673 | 0.9537 | 0.4065 | KRT18P28 |
| 0 | 0.6681 | 0.9837 | 0.4513 | LENG8 |
| 0 | 0.6681 | 0.9342 | 0.4758 | PHACTR4 |
| 0 | 0.6686 | 0.9637 | 0.2579 | GUSBL2 |
| 0 | 0.6687 | 0.9857 | 0.3898 | ACAD9 |
| 0 | 0.6695 | 0.9823 | 0.314 | ADORA3 |
| 0 | 0.6705 | 0.9952 | 0.3506 | MBNL1 |
| 0 | 0.6718 | 0.9308 | 0.3587 | C14orf1 |
| 0 | 0.6723 | 0.9928 | 0.2639 | GGA2 |
| 0 | 0.6727 | 0.9366 | 0.2944 | PCSK6 |
| 0 | 0.6727 | 0.9055 | 0.2316 | SLC25A5 |
| 0 | 0.6733 | 0.9629 | 0.2627 | C20orf144 |
| 0 | 0.6735 | 0.9881 | 0.3268 | HIBCH |
| 0 | 0.6742 | 0.9071 | 0.507 | KRTAP13-2 |
| 0 | 0.6742 | 0.9975 | 0.4199 | RND3 |
| 0 | 0.6751 | 0.9585 | 0.3661 | MYC |
| 0 | 0.6754 | 0.9252 | 0.3509 | PSMC5 |
| 0 | 0.6757 | 0.9927 | 0.3963 | ALG13 |
| 0 | 0.677 | 0.9799 | 0.472 | FRAG1 |
| 0 | 0.677 | 0.9922 | 0.4235 | VPRBP |
| 0 | 0.6772 | 0.9503 | 0.3955 | HSPBP1 |
| 0 | 0.6776 | 0.8631 | 0.2532 | HNRPAB |
| 0 | 0.6778 | 0.8877 | 0.4002 | CEACAM1 |
| 0 | 0.6785 | 0.9998 | 0.4248 | RPS2 |
| 0 | 0.6785 | 0.9782 | 0.3189 | CD63 |
| 0 | 0.6789 | 0.9996 | 0.2981 | TRABD |
| 0 | 0.68 | 0.8164 | 0.5369 | C1orf212 |
| 0 | 0.6805 | 0.9543 | 0.4227 | MKI67 |
| 0 | 0.6806 | 0.9661 | 0.384 | PLD3 |
| 0 | 0.6809 | 0.9294 | 0.4219 | SMARCD2 |
| 0 | 0.6814 | 0.9555 | 0.4695 | PTEN |
| 0 | 0.6822 | 0.9686 | 0.3218 | PIAS1 |
| 0 | 0.6824 | 0.9467 | 0.2595 | PRKD3 |
| 0 | 0.6826 | 0.9225 | 0.4275 | ELAVL4 |
| 0 | 0.683 | 0.8928 | 0.3574 | KIAA0310 |
| 0 | 0.6831 | 0.9722 | 0.4076 | GCGR |
| 0 | 0.6833 | 0.9901 | 0.4466 | MMD |
| 0 | 0.6841 | 0.9864 | 0.3263 | BTG2 |
| 0 | 0.6843 | 0.9552 | 0.4427 | GNPTG |
| 0 | 0.6844 | 0.9991 | 0.3038 | SNX11 |
| 0 | 0.6844 | 0.9304 | 0.4359 | UBR2 |
| 0 | 0.6854 | 0.9374 | 0.2301 | SMCP |
| 0 | 0.6855 | 0.9261 | 0.3341 | EMP2 |
| 0 | 0.6869 | 0.9749 | 0.3817 | ACTB |
| 0 | 0.6885 | 0.8771 | 0.4549 | ENPP1 |
| 0 | 0.6887 | 0.9139 | 0.3565 | FAM148A |
| 0 | 0.6887 | 0.9374 | 0.4136 | NUDCD3 |
| 0 | 0.6893 | 0.9416 | 0.3452 | RPL10 |
| 0 | 0.6895 | 0.9063 | 0.2827 | GGA2 |
| 0 | 0.6911 | 0.9949 | 0.2505 | CLPP |
| 0 | 0.6934 | 0.922 | 0.4453 | LOC642852 |
| 0 | 0.6934 | 0.9627 | 0.4467 | CNFN |
| 0 | 0.6937 | 0.9749 | 0.4044 | KIF2C |
| 0 | 0.6939 | 0.9652 | 0.3715 | POLDIP2 |
| 0 | 0.6943 | 0.9799 | 0.3867 | C20orf20 |
| 0 | 0.6948 | 0.9357 | 0.4145 | LAYN |
| 0 | 0.695 | 0.9938 | 0.4247 | MRPS12 |
| 0 | 0.695 | 0.9715 | 0.3227 | UBE2T |
| 0 | 0.6962 | 0.9383 | 0.2122 | ZNF225 |
| 0 | 0.6969 | 0.9185 | 0.2386 | HIST1H2AA |
| 0 | 0.697 | 0.982 | 0.3922 | SLC25A31 |
| 0 | 0.6973 | 0.9732 | 0.4419 | PHACTR4 |
| 0 | 0.6974 | 0.9251 | 0.5496 | UBE2C |
| 0 | 0.6978 | 0.8539 | 0.4938 | KIAA2013 |
| 0 | 0.6988 | 0.9634 | 0.526 | CD63 |
| 0 | 0.6989 | 0.976 | 0.3421 | EHD2 |
| 0 | 0.699 | 0.9151 | 0.3437 | THSD4 |
| 0 | 0.6991 | 0.9421 | 0.2866 | CENPF |
| 0 | 0.6993 | 0.8774 | 0.3055 | VEGFA |
| 0 | 0.6995 | 0.976 | 0.4145 | PPIA |
| 0 | 0.6997 | 0.9205 | 0.421 | KIAA1815 |
| 0 | 0.6997 | 0.9916 | 0.2685 | NME4 |
| 0 | 0.6998 | 0.9845 | 0.3316 | SNX11 |
| 0 | 0.7005 | 0.9193 | 0.4062 | TMEM19 |
| 0 | 0.7005 | 0.9378 | 0.4089 | CACNG7 |
| 0 | 0.7006 | 0.9797 | 0.2201 | C20orf67 |
| 0 | 0.7014 | 0.937 | 0.3042 | DHPS |
| 0 | 0.7015 | 0.9375 | 0.4427 | TMEM45B |
| 0 | 0.7018 | 0.9624 | 0.4117 | C16orf42 |
| 0 | 0.7021 | 0.9396 | 0.3035 | TMEM121 |
| 0 | 0.7026 | 0.9773 | 0.4756 | DRD2 |
| 0 | 0.7027 | 0.9797 | 0.2284 | FAM173B |
| 0 | 0.7029 | 0.9749 | 0.4154 | TMEM121 |
| 0 | 0.7032 | 0.9691 | 0.2979 | KRT18P28 |
| 0 | 0.7034 | 0.9682 | 0.3619 | KRTAP13-2 |
| 0 | 0.704 | 0.9634 | 0.4285 | MGAT4B |
| 0 | 0.7045 | 0.896 | 0.4424 | hCG_1642354 |
| 0 | 0.7047 | 0.9711 | 0.4327 | EPOR |
| 0 | 0.7049 | 0.9221 | 0.3743 | SELO |
| 0 | 0.7051 | 0.9618 | 0.3537 | RPS25 |
| 0 | 0.7052 | 0.8959 | 0.3767 | RCL1 |
| 0 | 0.7057 | 0.9958 | 0.3935 | SLAIN2 |
| 0 | 0.706 | 0.9118 | 0.2027 | ARL8A |
| 0 | 0.7071 | 0.9575 | 0.3423 | FAM110A |
| 0 | 0.7071 | 0.9793 | 0.3858 | GPR22 |
| 0 | 0.7075 | 0.9443 | 0.2604 | IGKV2-24 |
| 0 | 0.7076 | 0.9262 | 0.4323 | GSN |
| 0 | 0.708 | 0.9902 | 0.3368 | ERBB2IP |
| 0 | 0.708 | 0.8642 | 0.436 | TMEM97 |
| 0 | 0.7086 | 0.932 | 0.3669 | MGST3 |
| 0 | 0.7087 | 0.9934 | 0.3936 | MELK |
| 0 | 0.709 | 0.9772 | 0.4655 | SF3B3 |
| 0 | 0.7091 | 0.942 | 0.4158 | HDAC4 |
| 0 | 0.711 | 0.983 | 0.4759 | UBE2T |
| 0 | 0.7122 | 0.9998 | 0.4827 | PCSK6 |
| 0 | 0.7129 | 0.9708 | 0.4504 | PITPNC1 |
| 0 | 0.7141 | 0.9939 | 0.469 | LOC401397 |
| 0 | 0.7144 | 0.9806 | 0.2118 | MKI67 |
| 0 | 0.715 | 0.8717 | 0.4644 | EGFR |
| 0 | 0.7153 | 0.8907 | 0.4257 | LASS6 |
| 0 | 0.7153 | 0.9665 | 0.4881 | PCTK2 |
| 0 | 0.7155 | 0.9625 | 0.2872 | UQCR |
| 0 | 0.7157 | 0.9843 | 0.4601 | TBC1D9 |
| 0 | 0.7162 | 0.9271 | 0.4327 | HSPA8 |
| 0 | 0.7163 | 0.8728 | 0.4736 | LSMD1 |
| 0 | 0.7167 | 0.9993 | 0.3291 | LOC730275 |
| 0 | 0.7169 | 0.9736 | 0.2485 | CDC6 |
| 0 | 0.7172 | 0.9637 | 0.4451 | KRT7 |
| 0 | 0.7175 | 0.8705 | 0.4871 | SIAH2 |
| 0 | 0.7181 | 0.9807 | 0.3871 | LOC653391 |
| 0 | 0.7185 | 0.9998 | 0.3324 | SLC30A10 |
| 0 | 0.7187 | 0.91 | 0.3928 | FBXO25 |
| 0 | 0.7193 | 0.9777 | 0.3884 | FAM127A |
| 0 | 0.7199 | 0.9324 | 0.3211 | C16orf14 |
| 0 | 0.7199 | 0.9397 | 0.4944 | CSNK1A1 |
| 0 | 0.7204 | 0.998 | 0.3332 | ZNF225 |
| 0 | 0.721 | 0.9495 | 0.4755 | LOC100128062 |
| 0 | 0.7216 | 0.993 | 0.4682 | RAB22A |
| 0 | 0.7226 | 0.9953 | 0.3791 | GALNT10 |
| 0 | 0.7226 | 0.8765 | 0.5133 | BMP2K |
| 0 | 0.7227 | 0.9492 | 0.5545 | IGHA1 |
| 0 | 0.7227 | 0.968 | 0.5317 | LOC642852 |
| 0 | 0.7227 | 0.9944 | 0.5061 | SMARCD2 |
| 0 | 0.7233 | 0.9504 | 0.3534 | KIAA2013 |
| 0 | 0.7238 | 0.9173 | 0.4837 | HNRPA3 |
| 0 | 0.7238 | 0.9597 | 0.449 | SLC25A31 |
| 0 | 0.7239 | 0.9859 | 0.2638 | FLAD1 |
| 0 | 0.7241 | 0.9939 | 0.318 | MGC24125 |
| 0 | 0.7246 | 0.9829 | 0.4979 | MGC24125 |
| 0 | 0.725 | 0.9312 | 0.572 | KIAA0310 |
| 0 | 0.7251 | 0.9792 | 0.4393 | VDAC1 |
| 0 | 0.7252 | 0.9648 | 0.4113 | TMEM174 |
| 0 | 0.7256 | 0.9579 | 0.4541 | STEAP3 |
| 0 | 0.7265 | 0.963 | 0.366 | PCTK2 |
| 0 | 0.727 | 0.9992 | 0.4787 | RPLP0 |
| 0 | 0.7272 | 0.9074 | 0.4215 | H2AFY |
| 0 | 0.7281 | 0.9945 | 0.4109 | GALNT2 |
| 0 | 0.7282 | 0.9207 | 0.411 | URM1 |
| 0 | 0.7282 | 0.9215 | 0.2106 | C1orf93 |
| 0 | 0.7283 | 0.9244 | 0.4316 | PTEN |
| 0 | 0.7284 | 0.9758 | 0.4679 | C1QL2 |
| 0 | 0.7286 | 0.9269 | 0.4543 | HEATR3 |
| 0 | 0.7286 | 0.9906 | 0.4525 | PTPRA |
| 0 | 0.7304 | 0.9659 | 0.4943 | KIAA1815 |
| 0 | 0.7305 | 0.9643 | 0.3026 | PITPNC1 |
| 0 | 0.7311 | 0.9658 | 0.531 | SMG1 |
| 0 | 0.7313 | 0.887 | 0.5312 | STK11IP |
| 0 | 0.7314 | 0.9265 | 0.5148 | ISOC1 |
| 0 | 0.7321 | 0.9483 | 0.4796 | MRPS12 |
| 0 | 0.7324 | 0.9566 | 0.2202 | MALAT1 |
| 0 | 0.7328 | 0.9623 | 0.2481 | GNPTG |
| 0 | 0.733 | 0.9962 | 0.4347 | TRIB3 |
| 0 | 0.7345 | 0.928 | 0.4988 | ACBD6 |
| 0 | 0.7349 | 0.9197 | 0.4493 | BRD2 |
| 0 | 0.7354 | 0.9875 | 0.4886 | KRT17 |
| 0 | 0.7361 | 0.9868 | 0.3632 | EXO1 |
| 0 | 0.7367 | 0.9275 | 0.4796 | IGHV1-69 |
| 0 | 0.7372 | 0.9624 | 0.4535 | C1orf104 |
| 0 | 0.7375 | 0.9398 | 0.4891 | LOC400590 |
| 0 | 0.7378 | 0.9654 | 0.4863 | IL6ST |
| 0 | 0.7383 | 0.9381 | 0.3418 | UBR2 |
| 0 | 0.7394 | 0.9405 | 0.3804 | MGAT4B |
| 0 | 0.7395 | 0.9226 | 0.5431 | UQCR |
| 0 | 0.7401 | 0.9929 | 0.3967 | NEDD8 |
| 0 | 0.7412 | 0.9511 | 0.5199 | ARHGEF11 |
| 0 | 0.7417 | 0.9628 | 0.3511 | MCCD1 |
| 0 | 0.7417 | 0.9515 | 0.4353 | MSI2 |
| 0 | 0.7421 | 0.9273 | 0.5576 | EMP2 |
| 0 | 0.7422 | 0.9362 | 0.6205 | MARVELD2 |
| 0 | 0.7422 | 0.9361 | 0.5301 | MYO1F |
| 0 | 0.7433 | 0.9757 | 0.453 | C1orf93 |
| 0 | 0.7434 | 0.9692 | 0.4942 | ORMDL3 |
| 0 | 0.7438 | 0.9593 | 0.4619 | UGCG |
| 0 | 0.7441 | 0.9964 | 0.506 | MGC70870 |
| 0 | 0.7444 | 0.9194 | 0.3783 | ATXN2 |
| 0 | 0.7448 | 0.967 | 0.3434 | ADCY7 |
| 0 | 0.745 | 0.9971 | 0.5858 | CEP55 |
| 0 | 0.7455 | 0.9163 | 0.5755 | LSMD1 |
| 0 | 0.7455 | 0.8844 | 0.5856 | PXN |
| 0 | 0.7462 | 0.9987 | 0.5026 | SMCP |
| 0 | 0.7463 | 0.9602 | 0.5405 | SNX5 |
| 0 | 0.7476 | 0.9991 | 0.3192 | C20orf20 |
| 0 | 0.7482 | 0.8923 | 0.4533 | CYB561 |
| 0 | 0.7483 | 0.9933 | 0.2156 | VPS45A |
| 0 | 0.7484 | 0.9947 | 0.4546 | LOC649178 |
| 0 | 0.7485 | 0.9019 | 0.4989 | MAP3K13 |
| 0 | 0.7492 | 0.9644 | 0.5197 | SLC16A8 |
| 0 | 0.7498 | 0.9895 | 0.2785 | TAPBP |
| 0 | 0.75 | 0.8713 | 0.3743 | ZNF592 |
| 0 | 0.7501 | 0.9806 | 0.5118 | ARFGEF2 |
| 0 | 0.7503 | 0.9745 | 0.2555 | CIZ1 |
| 0 | 0.7507 | 0.9536 | 0.3272 | FLJ35390 |
| 0 | 0.7507 | 0.9894 | 0.5138 | ADAMTSL5 |
| 0 | 0.7508 | 0.9461 | 0.5709 | IGH@ |
| 0 | 0.7508 | 0.9937 | 0.3778 | NLK |
| 0 | 0.751 | 0.9693 | 0.4256 | MCCD1 |
| 0 | 0.7511 | 0.9154 | 0.5499 | LOC652261 |
| 0 | 0.7511 | 0.9991 | 0.371 | ATAD3A |
| 0 | 0.7514 | 0.987 | 0.4115 | LOC442019 |
| 0 | 0.7514 | 0.9624 | 0.4926 | RHBDD1 |
| 0 | 0.752 | 0.9436 | 0.3224 | FKSG30 |
| 0 | 0.7521 | 0.9559 | 0.5952 | CXorf56 |
| 0 | 0.7523 | 0.9819 | 0.4708 | RPAP1 |
| 0 | 0.7525 | 0.9973 | 0.5524 | ELAVL4 |
| 0 | 0.7526 | 0.9579 | 0.4707 | CLNS1A |
| 0 | 0.7528 | 0.9443 | 0.4462 | TYMS |
| 0 | 0.7529 | 0.9963 | 0.5272 | C8orf73 |
| 0 | 0.7534 | 0.953 | 0.3571 | KHSRP |
| 0 | 0.7536 | 0.9868 | 0.5323 | KRTAP5-9 |
| 0 | 0.7538 | 0.9801 | 0.3808 | FAM83E |
| 0 | 0.7538 | 0.941 | 0.4053 | VPRBP |
| 0 | 0.7543 | 0.9004 | 0.6412 | MBOAT2 |
| 0 | 0.7549 | 0.9813 | 0.2649 | CEP55 |
| 0 | 0.7551 | 0.9893 | 0.243 | LCE3E |
| 0 | 0.7551 | 0.9603 | 0.4614 | MLPH |
| 0 | 0.7556 | 0.9967 | 0.3562 | KIAA0146 |
| 0 | 0.7561 | 0.961 | 0.2536 | CACNG7 |
| 0 | 0.7562 | 0.952 | 0.3203 | GALNT10 |
| 0 | 0.7564 | 0.9683 | 0.2773 | EHD2 |
| 0 | 0.7569 | 0.9906 | 0.5289 | CXXC5 |
| 0 | 0.7571 | 0.9337 | 0.5152 | CD63 |
| 0 | 0.7573 | 0.9824 | 0.337 | JMJD1B |
| 0 | 0.7576 | 0.957 | 0.4758 | FRAG1 |
| 0 | 0.7576 | 0.9839 | 0.5753 | TRABD |
| 0 | 0.7581 | 0.9954 | 0.4265 | NAT10 |
| 0 | 0.7591 | 0.9745 | 0.4933 | MAFK |
| 0 | 0.7607 | 0.978 | 0.3086 | POLR2L |
| 0 | 0.761 | 0.9127 | 0.506 | HRH2 |
| 0 | 0.7622 | 0.984 | 0.4022 | KIAA0652 |
| 0 | 0.7622 | 0.9742 | 0.4243 | CLNS1A |
| 0 | 0.7628 | 0.9515 | 0.5061 | REPS2 |
| 0 | 0.7635 | 0.9124 | 0.4856 | TBX21 |
| 0 | 0.7642 | 0.9801 | 0.4484 | MED13L |
| 0 | 0.7649 | 0.9628 | 0.4702 | DDX50 |
| 0 | 0.765 | 0.9625 | 0.4859 | EHMT1 |
| 0 | 0.765 | 0.9878 | 0.3723 | MLPH |
| 0 | 0.766 | 0.9948 | 0.4551 | POLR3H |
| 0 | 0.7661 | 0.9813 | 0.4048 | EVL |
| 0 | 0.7663 | 0.9967 | 0.389 | POGZ |
| 0 | 0.7664 | 0.9321 | 0.5645 | KRTAP5-9 |
| 0 | 0.7666 | 0.9956 | 0.3713 | CDC20 |
| 0 | 0.7669 | 0.9805 | 0.4749 | HDGFRP3 |
| 0 | 0.7672 | 0.9786 | 0.3668 | DOT1L |
| 0 | 0.7672 | 0.9904 | 0.3309 | SLAIN2 |
| 0 | 0.7673 | 0.9873 | 0.5417 | NUF2 |
| 0 | 0.7677 | 0.9881 | 0.5894 | SLC25A28 |
| 0 | 0.7685 | 0.9939 | 0.409 | KIAA2013 |
| 0 | 0.7685 | 0.9925 | 0.4487 | SMG1 |
| 0 | 0.7688 | 0.9988 | 0.4309 | PPIA |
| 0 | 0.7692 | 0.9851 | 0.4793 | MSN |
| 0 | 0.7706 | 0.9768 | 0.4737 | HNRPAB |
| 0 | 0.7708 | 0.9968 | 0.505 | DOT1L |
| 0 | 0.7708 | 0.9438 | 0.4485 | BRAF |
| 0 | 0.7709 | 0.9432 | 0.5775 | ROMO1 |
| 0 | 0.7709 | 0.9916 | 0.4307 | ACTB |
| 0 | 0.771 | 0.9931 | 0.3584 | MAZ |
| 0 | 0.771 | 0.996 | 0.4555 | RHBDD1 |
| 0 | 0.7711 | 0.9295 | 0.605 | GSTM1 |
| 0 | 0.7715 | 0.9934 | 0.482 | BCL2 |
| 0 | 0.7716 | 0.9675 | 0.559 | NDUFB9 |
| 0 | 0.7718 | 0.9952 | 0.324 | UGDH |
| 0 | 0.7723 | 0.9955 | 0.3569 | ZACN |
| 0 | 0.7727 | 0.9836 | 0.5241 | TAF2 |
| 0 | 0.7735 | 0.9948 | 0.4459 | MELK |
| 0 | 0.7737 | 0.9772 | 0.5689 | ERBB4 |
| 0 | 0.7744 | 0.9909 | 0.437 | NLK |
| 0 | 0.7755 | 0.9979 | 0.4124 | RPS2 |
| 0 | 0.7755 | 0.955 | 0.498 | CDH3 |
| 0 | 0.7757 | 0.9979 | 0.3835 | ZNF704 |
| 0 | 0.7758 | 0.987 | 0.4859 | ZNF609 |
| 0 | 0.776 | 0.948 | 0.6177 | VPS45A |
| 0 | 0.7763 | 0.998 | 0.3259 | C20orf144 |
| 0 | 0.7777 | 0.9665 | 0.2782 | C1orf104 |
| 0 | 0.7785 | 0.9589 | 0.4912 | RPAP1 |
| 0 | 0.7786 | 0.9215 | 0.4194 | IGKV2-24 |
| 0 | 0.7788 | 0.9928 | 0.5772 | JMJD1B |
| 0 | 0.7789 | 0.9847 | 0.4949 | KRTAP19-1 |
| 0 | 0.7789 | 0.9834 | 0.4657 | MCCD1 |
| 0 | 0.7793 | 0.9677 | 0.6465 | UBE2W |
| 0 | 0.7794 | 0.9996 | 0.4779 | SLC7A2 |
| 0 | 0.7798 | 0.959 | 0.4224 | CIZ1 |
| 0 | 0.7801 | 0.9777 | 0.5373 | CRTC2 |
| 0 | 0.7806 | 0.9715 | 0.3712 | FARP2 |
| 0 | 0.7809 | 0.9938 | 0.5772 | KRTAP19-1 |
| 0 | 0.7811 | 0.9545 | 0.5695 | FGFR4 |
| 0 | 0.7813 | 0.9954 | 0.4387 | CYBRD1 |
| 0 | 0.7814 | 0.932 | 0.6018 | LOC652261 |
| 0 | 0.7818 | 0.9813 | 0.5384 | TBXAS1 |
| 0 | 0.7822 | 0.9972 | 0.4964 | UHMK1 |
| 0 | 0.7824 | 0.9583 | 0.5898 | ASPHD2 |
| 0 | 0.7831 | 0.9568 | 0.6717 | KRT5 |
| 0 | 0.7832 | 0.9533 | 0.4823 | IRGC |
| 0 | 0.7835 | 0.9207 | 0.5815 | EVL |
| 0 | 0.7835 | 0.9866 | 0.344 | FOXA1 |
| 0 | 0.7835 | 0.9765 | 0.5165 | ZNF592 |
| 0 | 0.7852 | 0.9288 | 0.4759 | CDH3 |
| 0 | 0.7854 | 0.9934 | 0.5758 | GPRIN1 |
| 0 | 0.7857 | 0.9679 | 0.5555 | NUDCD3 |
| 0 | 0.7859 | 0.9042 | 0.5553 | ARFGEF2 |
| 0 | 0.7864 | 0.9803 | 0.4765 | L3MBTL2 |
| 0 | 0.7874 | 0.9729 | 0.5098 | FAM127A |
| 0 | 0.7875 | 0.9916 | 0.4092 | FOXC1 |
| 0 | 0.7884 | 0.9889 | 0.4946 | MYO1F |
| 0 | 0.7887 | 0.9734 | 0.6167 | ENO1 |
| 0 | 0.7887 | 0.995 | 0.4453 | IFI27L1 |
| 0 | 0.7893 | 0.9625 | 0.2405 | GNPTG |
| 0 | 0.7893 | 0.9634 | 0.485 | HDAC4 |
| 0 | 0.7907 | 0.9676 | 0.5618 | C15orf52 |
| 0 | 0.7908 | 0.9863 | 0.3637 | SCUBE2 |
| 0 | 0.791 | 0.9069 | 0.414 | ERBB2IP |
| 0 | 0.7912 | 0.9935 | 0.5062 | VPS18 |
| 0 | 0.7924 | 0.9953 | 0.4164 | TMCO1 |
| 0 | 0.7927 | 0.9936 | 0.5016 | UQCR |
| 0 | 0.7928 | 0.9551 | 0.481 | DOT1L |
| 0 | 0.794 | 0.9783 | 0.511 | FAM110A |
| 0 | 0.7942 | 0.9611 | 0.5636 | ADCYAP1 |
| 0 | 0.7942 | 0.9928 | 0.3832 | GSR |
| 0 | 0.7943 | 0.9775 | 0.561 | FAM173B |
| 0 | 0.7944 | 0.9635 | 0.3841 | PIAS1 |
| 0 | 0.7946 | 0.9932 | 0.563 | NAT10 |
| 0 | 0.7948 | 0.9923 | 0.2763 | MAP3K13 |
| 0 | 0.7949 | 0.9865 | 0.2372 | KIAA1920 |
| 0 | 0.7955 | 0.9871 | 0.4835 | LOC100128062 |
| 0 | 0.7956 | 0.9578 | 0.6056 | CIZ1 |
| 0 | 0.7961 | 0.992 | 0.4024 | CDC20 |
| 0 | 0.7966 | 0.9016 | 0.5901 | POLD4 |
| 0 | 0.7978 | 0.9634 | 0.4638 | LARS |
| 0 | 0.7978 | 0.9967 | 0.5465 | ARHGEF11 |
| 0 | 0.7982 | 0.9963 | 0.4248 | HEATR3 |
| 0 | 0.7988 | 0.9866 | 0.5443 | ATXN2 |
| 0 | 0.7998 | 0.9941 | 0.512 | TAF2 |
| 0 | 0.8001 | 0.9855 | 0.4593 | HIST1H2AA |
| 0 | 0.8001 | 0.9997 | 0.5497 | PHB2 |
| 0 | 0.8002 | 0.9594 | 0.5511 | MARVELD2 |
| 0 | 0.8006 | 0.9752 | 0.5392 | FKSG30 |
| 0 | 0.8011 | 0.9741 | 0.527 | PIAS1 |
| 0 | 0.8012 | 0.9275 | 0.701 | ERBB4 |
| 0 | 0.8018 | 0.9315 | 0.5793 | SCUBE2 |
| 0 | 0.8028 | 0.9998 | 0.3789 | GUSB |
| 0 | 0.8029 | 0.9209 | 0.6017 | MFSD1 |
| 0 | 0.8032 | 0.9991 | 0.3723 | CENPF |
| 0 | 0.8033 | 0.9838 | 0.5098 | ADCYAP1 |
| 0 | 0.8039 | 0.9871 | 0.5352 | KRT5 |
| 0 | 0.8045 | 0.9884 | 0.4947 | NUDCD3 |
| 0 | 0.8045 | 0.98 | 0.6506 | SLC25A28 |
| 0 | 0.8045 | 0.9543 | 0.6513 | SMS |
| 0 | 0.8051 | 0.9972 | 0.4344 | TBXAS1 |
| 0 | 0.8055 | 0.9735 | 0.5294 | MGC4093 |
| 0 | 0.8056 | 0.9717 | 0.6619 | PLEKHF2 |
| 0 | 0.8057 | 0.9784 | 0.4543 | IRGC |
| 0 | 0.8066 | 0.9421 | 0.5289 | FKSG30 |
| 0 | 0.8074 | 0.9622 | 0.6389 | NAT10 |
| 0 | 0.8077 | 0.9586 | 0.4516 | PHB2 |
| 0 | 0.8079 | 0.9646 | 0.5062 | SFRS1 |
| 0 | 0.8081 | 0.9591 | 0.4882 | PHGDH |
| 0 | 0.8085 | 0.9918 | 0.5035 | CNFN |
| 0 | 0.8087 | 0.9482 | 0.5376 | POLD4 |
| 0 | 0.8087 | 0.975 | 0.5217 | ADAMTSL5 |
| 0 | 0.809 | 0.9991 | 0.3924 | IGJ |
| 0 | 0.8092 | 0.9619 | 0.5416 | BIRC5 |
| 0 | 0.8094 | 0.9991 | 0.5216 | LOC643159 |
| 0 | 0.8102 | 0.9987 | 0.5383 | CSNK1A1 |
| 0 | 0.8105 | 0.9991 | 0.645 | PLD4 |
| 0 | 0.8114 | 0.9584 | 0.6001 | FLJ22659 |
| 0 | 0.8118 | 0.9781 | 0.5185 | MYC |
| 0 | 0.8125 | 0.9773 | 0.4933 | Kua-UEV |
| 0 | 0.8126 | 0.9912 | 0.4778 | LOC643159 |
| 0 | 0.8126 | 0.9523 | 0.6253 | PDZK1IP1 |
| 0 | 0.8132 | 0.9899 | 0.5081 | MAD2L2 |
| 0 | 0.8145 | 0.9697 | 0.54 | MSN |
| 0 | 0.8146 | 0.995 | 0.5387 | FABP5 |
| 0 | 0.816 | 0.9743 | 0.4729 | NME4 |
| 0 | 0.8166 | 0.9423 | 0.4208 | ANAPC1 |
| 0 | 0.8173 | 0.9896 | 0.5609 | METTL3 |
| 0 | 0.8177 | 0.9082 | 0.582 | FAM173B |
| 0 | 0.8191 | 0.9887 | 0.5152 | MYC |
| 0 | 0.8191 | 0.9742 | 0.5227 | CCDC74A |
| 0 | 0.8192 | 0.9726 | 0.5983 | GHR |
| 0 | 0.8198 | 0.95 | 0.5253 | OGFR |
| 0 | 0.82 | 0.985 | 0.6715 | CD68 |
| 0 | 0.8202 | 0.973 | 0.657 | HNRPA3 |
| 0 | 0.8203 | 0.9702 | 0.4825 | PPP2R2D |
| 0 | 0.8205 | 0.9733 | 0.5151 | GSR |
| 0 | 0.8206 | 0.9986 | 0.5146 | ADCY7 |
| 0 | 0.8212 | 0.9688 | 0.5202 | SMS |
| 0 | 0.8224 | 0.9819 | 0.6383 | FARP2 |
| 0 | 0.823 | 0.9939 | 0.5167 | MVP |
| 0 | 0.8237 | 0.9959 | 0.6136 | ARFGEF2 |
| 0 | 0.8257 | 0.9757 | 0.6619 | GUSBL2 |
| 0 | 0.8259 | 0.9843 | 0.6225 | CLIP3 |
| 0 | 0.8264 | 0.9883 | 0.6808 | HRH2 |
| 0 | 0.8268 | 0.9875 | 0.6082 | CD68 |
| 0 | 0.8275 | 0.9936 | 0.6562 | LOC400590 |
| 0 | 0.8275 | 0.9781 | 0.599 | RRM2 |
| 0 | 0.8278 | 0.9674 | 0.5771 | NEBL |
| 0 | 0.8282 | 0.9683 | 0.5229 | TMEM19 |
| 0 | 0.8287 | 0.9992 | 0.5074 | SULT1A2 |
| 0 | 0.8295 | 0.9874 | 0.4886 | PCSK6 |
| 0 | 0.8301 | 0.9704 | 0.611 | C14orf1 |
| 0 | 0.8306 | 0.9989 | 0.5225 | AGPS |
| 0 | 0.8308 | 0.9686 | 0.4749 | FLJ22795 |
| 0 | 0.8317 | 0.959 | 0.6623 | LOC442019 |
| 0 | 0.8322 | 0.9763 | 0.6096 | TMCO1 |
| 0 | 0.8331 | 0.9889 | 0.6527 | FLJ35390 |
| 0 | 0.8334 | 0.995 | 0.5498 | KRT17 |
| 0 | 0.8341 | 0.9754 | 0.5523 | SNX5 |
| 0 | 0.8343 | 0.9954 | 0.4872 | C20orf20 |
| 0 | 0.8347 | 0.9954 | 0.4962 | FOXC1 |
| 0 | 0.8347 | 0.9672 | 0.7422 | GSR |
| 0 | 0.8355 | 0.9891 | 0.6917 | EVL |
| 0 | 0.837 | 0.9727 | 0.5342 | LOC200810 |
| 0 | 0.8384 | 0.9918 | 0.6627 | BMP2K |
| 0 | 0.8387 | 0.9783 | 0.6471 | DPY19L4 |
| 0 | 0.8415 | 0.9997 | 0.6035 | SULT1A2 |
| 0 | 0.8423 | 0.9859 | 0.6327 | TMBIM1 |
| 0 | 0.843 | 0.999 | 0.6451 | ANAPC1 |
| 0 | 0.845 | 0.9743 | 0.6192 | LOC653391 |
| 0 | 0.8455 | 0.9924 | 0.709 | PRELID1 |
| 0 | 0.846 | 0.9775 | 0.6775 | MVP |
| 0 | 0.8465 | 0.9855 | 0.5448 | ETS2 |
| 0 | 0.8468 | 0.9994 | 0.7006 | HNRPA3 |
| 0 | 0.847 | 0.9761 | 0.5791 | CLPB |
| 0 | 0.8482 | 0.9834 | 0.4922 | MGAT4B |
| 0 | 0.8497 | 0.9957 | 0.5635 | IMPAD1 |
| 0 | 0.8501 | 0.9438 | 0.6114 | PHRF1 |
| 0 | 0.851 | 0.9683 | 0.66 | VPS18 |
| 0 | 0.8523 | 0.9642 | 0.6895 | SELO |
| 0 | 0.8528 | 0.9957 | 0.6256 | GHR |
| 0 | 0.8538 | 0.9849 | 0.7121 | PHGDH |
| 0 | 0.8547 | 0.9979 | 0.6107 | PLD4 |
| 0 | 0.8585 | 0.958 | 0.7646 | ZNF592 |
| 0 | 0.8601 | 0.9967 | 0.6851 | MYO1F |
| 0 | 0.8602 | 0.9796 | 0.5052 | OGFR |
| 0 | 0.8603 | 0.9876 | 0.712 | LOC200810 |
| 0 | 0.8608 | 0.9784 | 0.5134 | RASD2 |
| 0 | 0.8671 | 0.9935 | 0.6818 | PLEKHF2 |
| 0 | 0.8677 | 0.9848 | 0.6755 | FLJ22795 |
| 0 | 0.8685 | 0.9998 | 0.5926 | PTPRA |
| 0 | 0.8697 | 0.988 | 0.7056 | BAG1 |
| 0 | 0.8708 | 0.997 | 0.6107 | ATXN2 |
| 0 | 0.871 | 0.9679 | 0.4552 | ANAPC1 |
| 0 | 0.8727 | 0.9988 | 0.5969 | RELB |
| 0 | 0.8732 | 0.9981 | 0.7045 | CD68 |
| 0 | 0.8755 | 0.9856 | 0.7283 | TRIB3 |
| 0 | 0.8776 | 0.9917 | 0.6808 | SLC25A29 |
| 0 | 0.8807 | 0.9743 | 0.6968 | GRB7 |
| 0 | 0.8855 | 0.9921 | 0.6874 | STK11IP |
| 0 | 0.8868 | 0.9881 | 0.6278 | ALG13 |
| 0 | 0.8956 | 0.9952 | 0.6646 | NEDD8 |
| 0 | 0.9076 | 0.9843 | 0.7082 | RELB |

Since each gene was treated as categorical variable based on quartiles with lowest quartile as reference, there are three categories for each gene. Mean, minimum, and maximum interaction p-values from 10-fold jack knifing process are shown. Fifteen genes were significant 100% of the time (FLOT2, CA12, TUBB2C, UNC119, GATA3, SUPT6H, RPL23A, SLC39A14, ABHD2, FTH1, FAM84B, ACVR1B, ZACN). Clustering of these or any other combination of genes selected purely based on statistical significance did not allow for robust identification of subsets with differential benefit from trastuzumab. In light of this, it was decided to attempt an additional approach to identify subsets with differential benefit from trastuzumab.

From among all of the results of gene assessment performed, it was noticed that the top predictive genes included several estrogen receptor associated genes, CA12 (mean interaction p=0.0059), GATA3 (p=0.007), PIK3A (p=0.0388) as well as genes from HER2 amplicon: ERBB2 (p=0.0485) and C17orf37 (p=0.0442). Using this information and the facts that ER status has been associated with lower rates of complete pathological response in several published studies (Untch, et al., supra; Bhargava, et al., Mod. Pathol. 24:367-374, 2011) and that HER2 (ERBB2) is the target for trastuzumab, it was decided to select, as the basis to develop a predictive algorithm, genes whose expression levels were correlated with ESR1 mRNA or with ERBB2 mRNA having Spearman's correlation coefficient over 0.7 and also a minimum interaction P value below 0.1. The top genes correlated with ESR1 and ERBB2 are shown in Table 5. From this pool, 8 genes met the criteria of a correlation coefficient over 0.7 and a minimum interaction P value below 0.1. These genes included ESR1, NAT1, GATA3, CA12, IGFR1, ERBB2, c17orf37 and GRB7.

TABLE 5

| | | Minimum |
|---|---|---|
| Gene | | Interaction |
| Symbol | | P Value |
| | Correlation with | |
| | ERBB2 | |
| ERBB2 | 1 | 0.025 |
| GRB7 | 0.912 | 0.06 |
| C17orf37 | 0.833 | 0.0003 |
| KRT7 | 0.498 | 0.047 |
| TMEM45B | 0.453 | 0.29 |
| ORMDL3 | 0.448 | 0.076 |
| C1orf93 | 0.427 | 0.1 |
| SPDEF | 0.4 | 0.013 |
| VEGFA | 0.395 | 0.24 |
| FGFR4 | 0.347 | 0.35 |
| | Correlation with | |
| | ESR1 | |
| ESR1 | 1 | 0.064 |
| TBC1D9 | 0.757 | 0.49 |
| CA12 | 0.733 | 0.0024 |
| IGF1R | 0.731 | 0.042 |
| GATA3 | 0.727 | 0.0036 |
| THSD4 | 0.727 | 0.12 |
| NAT1 | 0.701 | 0.075 |
| SLC39A6 | 0.685 | 0.21 |
| SCUBE2 | 0.637 | 0.47 |
| SIAH2 | 0.632 | 0.19 |

In order to identify subsets with different degree of benefit from trastuzumab while accommodating the non-linearity of interaction between genes and trastuzumab, the first two principal components (PC1 and PC2) obtained from the 8 selected predictive genes were used to create a three dimensional subset treatment effect pattern plot with spline interpolation to smooth the plot with hazard ratio for trastuzumab on Z-axis. Hazard ratios were color coded as green if less than 0.5 (large benefit from trastuzumab), brown for 0.5-1.0 (moderate benefit), or red for over 1.0 (no benefit). This plot readily identified subsets with differential benefit from trastuzumab. Cut-points were derived for two principal components (PC1 and PC2) that defined three subsets based on TDSTEPP and the event rate in each subgroup.

The cut-points for two principal components (PC1 and PC2) that defined these three subsets were determined as follows: No benefit group if PC1>0.6 and PC2>0.1; Large benefit group if −0.12<PC1<=0.6 and 0.1<PC2<=0.6 and PC2>PC1+0.22, if −0.6<PC1<=0.6 and PC2>=0.6, or if PC1<=−0.12 and −0.55<PC2<0.6. Remaining patients were classified as the moderate benefit group.

Kaplan-Meier plots were created for three subsets identified using these cut-points for PC1 and PC2. The no benefit group (Group 1, N=81) had a hazard ratio of 1.56. The moderate benefit group (Group 2, N=255) had a HR of 0.56, and the large benefit group (Group 3, N=252) had a HR of 0.27. It should be noted that p-values and confidence intervals for these data are not appropriate, because these plots are for the discovery cohort that was used to develop the algorithm. The plots were used to illustrate the degree of differentiation in trastuzumab effect that is achieved with the algorithm.

Assessment of the Pre-Defined Cut-Points for the Prediction Model in the Confirmation Cohort

The pre-defined cut-points from the 8-gene prediction model described above were assessed in the remaining 991 B-31 patients not included in the discovery phase for whom specimens were available. Since the algorithm has not yet been developed into a formal clinical test, a formal NCI registered date stamped protocol was not developed before proceeding to the cut-points assessment. Kaplan-Meier plots were created based on the pre-defined cut-off values for the two principal components created by applying the eigen-vector coefficients from the candidate discovery set to the confirmation dataset. Applying the pre-defined cut-points for the 8-gene prediction model readily identified: a subset with no benefit from trastuzumab (Group 1) with a hazard ratio of 1.58 (95% CI: 0.67-3.69, p=0.29, N=100), a subset with moderate benefit (Group 2) with a hazard ratio of 0.60 (95% CI: 0.41-0.89, p=0.011, N=449), and a subset with large benefit (Group 3) with a hazard ratio of 0.28 (95% CI: 0.20-0.41, p<0.0001, N=442). The p-value for the interaction between predictive algorithm and trastuzumab was 0.0002.

Distribution of Central HER2Assay Negative Cases Among Categories Defined by the Prediction Model

Because HER2 is the target for trastuzumab, it is expected that Group 1 with no benefit should express the lowest levels of ERBB2 mRNA. A correlation analysis was performed between ERBB2 and ESR1 mRNA levels in which each subgroup defined by the 8-gene prediction model. Surprisingly, the subset with no benefit expressed high levels of ESR1 mRNA and intermediate levels of ERBB2 mRNA rather than the lowest levels in both candidate discovery and confirmation cohorts.

An unexpected finding from the B-31 trial was that central HER2 assay negative patients also derived benefit from trastuzumab. Because the 8-gene prediction model was developed independent of the knowledge of centrally performed HER2 testing results, it was tested whether central HER2 assay negative cases belong to the Group 1 defined by the predictive model with no expected benefit. When central HER2 negative results were overlaid on these subsets, only a few HER2 negative patients belonged to the subgroup with no benefit, while a majority belonged to the moderate-benefit subgroup.

These results support the hypothesis that HER2 negative patients may derive benefit from trastuzumab.

Discussion

Using multiplexed gene expression profiling with RNA extracted from archived formalin fixed paraffin embedded tumor blocks from NSABP trial B-31, a predictive algorithm for the degree of benefit from trastuzumab added to adjuvant chemo-endocrine therapy of HER2 positive breast cancer was developed. In the internal confirmation set of 991 patients, this algorithm and pre-defined cut-points were validated with interaction p-value of 0.0002.

The data demonstrate a complex relationship between HER2 and ER as determinants of clinical benefit from trastuzumab added to adjuvant chemo-endocrine therapy. ERBB2 mRNA-by-trastuzumab interaction was not linear and was also modulated by other genes, especially those from estrogen receptor pathway. Most surprisingly, the identified subgroup with no clinical benefit from adjuvant trastuzumab actually expressed intermediate—not the lowest—levels of ERBB2 mRNA, together with the highest levels of ESR1-associated genes. This subgroup also had an excellent baseline prognosis, which was similar to the prognosis of others treated with trastuzumab.

While not bound to any particular theory, there could be at least two explanations for the lack of benefit in this subgroup. In NSABP trial B-14, it was observed that ESR1 mRNA level is a linear predictor of the degree of benefit from tamoxifen (Kim, et al., J. Clin. Oncol. 29:4160-4167, 2011). Therefore, one explanation may be that patients with tumors that express highest levels of ESR1 and its associated mRNAs may have already derived maximum clinical benefit from antiestrogen therapy. An alternative explanation is that such tumors are biologically resistant to trastuzumab. Lower rate of complete pathological response to neoadjuvant trastuzumab in ER-positive tumors compared to ER-negative tumors supports the second interpretation. It is possible that estrogen receptor is directly responsible by inducing anti-apoptotic proteins such as Bcl-2 or IGF1R. Overexpressed IGF1R can hetero-trimerize with HER2 and EGFR, and cause resistance to trastuzumab in vitro and in vivo (Huang, et al., Cancer Res. 70:1204-1214, 2010; Lu, et al., J. Natl. Cancer Inst. 93:1852-1857, 2001). In reality, due to a close association of expression levels among these genes, it is impossible to separate them.

Regardless of the mechanisms responsible for no clinical benefit, therapeutic strategies to improve the outcome of this subgroup need to be developed because, although their prognosis is favorable, patients still suffer from over 10% recurrences in 5 years, which is not improved by the addition of trastuzumab. A combination of HER2, ER, and IGF1R targeting, HER2 targeting combined with complete blockage of ER pathway using fulvestrant (because IGF1R is induced by ER; Osborne, et al., Br. J. Cancer 90 Suppl. 1:S2-S6, 2004), or a SRC inhibitor (Zhang, et al., Nat. Med. 17:461-469, 2011) may be a potential strategy.

The data also support the hypothesis based on central HER2 testing results from B-31 that HER2 negative patients may benefit from adjuvant trastuzumab. Because HER2 negative patients belong to Group 2, approximately 40 percent reduction in recurrences is expected from the addition of trastuzumab to adjuvant chemotherapy with minor side effects. This hypothesis is currently being tested through a randomized clinical trial (NSABP protocol B-47: NCT01275677).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method of identifying a cancer patient that has an increased benefit from the addition of a HER2-targeted therapy to a standard chemotherapy regimen, comprising assaying a tumor tissue sample from said patient for expression of HER2 or a HER2-related mRNA and estrogen receptor or an estrogen receptor-related mRNA, wherein a value outside of a range of a combined normalized HER2 mRNA expression level between about 11.0 and about 15.0 and a normalized estrogen receptor mRNA expression level of about 10.0 and about 12.0 is indicative of a cancer patient that has an increased benefit from the addition of a HER2-targeted therapy to a chemotherapy regimen.

2. The method of claim 1, wherein the HER2-targeted therapy is trastuzumab.

3. The method of claim 1, wherein the cancer is breast cancer.

4. The method of claim 3, wherein the chemotherapy regimen involves the administration of 4 cycles of doxorubicin plus cyclophosphamide followed by 4 cycles of paclitaxel to the cancer patient.

5. The method of claim 1, wherein the HER2-related mRNA is a c17orf37 or GRB7 mRNA.

6. The method of claim 1, wherein the estrogen receptor-related mRNA is a NAT1, GATA3, CA12 or IGF1R mRNA.

7. A method of treating breast cancer in a patient in need of such treatment, comprising:
a) assaying a tumor tissue sample from said patient for expression of HER2 or a HER2-related mRNA and estrogen receptor or an estrogen receptor-related mRNA; and
b) treating the patient with a HER2-targeted therapy and a chemotherapy regimen if the results of the assay indicate a value outside of a range of a combined normalized HER2 or HER2-related mRNA expression level between about 11.0 and about 15.0 and a normalized estrogen receptor or estrogen receptor-related mRNA expression level of about 10.0 and about 12.0.

8. The method of claim 7, wherein the HER2-targeted therapy is trastuzumab.

9. The method of claim 7, wherein the chemotherapy regimen involves the administration of 4 cycles of doxorubicin plus cyclophosphamide followed by 4 cycles of paclitaxel to the breast cancer patient.

10. The method of claim 7, wherein the HER2-related mRNA is a c17orf37 or GRB7 mRNA.

11. The method of claim 7, wherein the estrogen receptor-related mRNA is a NAT1, GATA3, CA12 or IGF1R mRNA.
